(19) United States
(12) Patent Application Publication
(10) Pub. No.: US 2024/0404056 A1
(43) Pub. Date: Dec. 5, 2024

(54) WALL THICKNESS ESTIMATION METHOD, RECORDING MEDIUM, TRAINING METHOD, MODEL CONSTRUCTION METHOD, WALL THICKNESS ESTIMATION DEVICE, AND WALL THICKNESS ESTIMATION SYSTEM

(71) Applicant: OSAKA UNIVERSITY, Osaka (JP)

(72) Inventor: Yoshie SUGIYAMA, Osaka (JP)

(21) Appl. No.: 18/696,454

(22) PCT Filed: Oct. 7, 2022
(86) PCT No.: PCT/JP2022/037678

§ 371 Date: Mar. 28, 2024

(30) Foreign Application Priority Data
Oct. 8, 2021 (JP) 2021-166437

(51) Int. Cl. G06T 7/00; G06T 7/62

(52) U.S. Cl. CPC G06T 7/0012; G06T 7/62; G06T 2207/10016; G06T 2207/20081; G06T 2207/30016; G06T 2207/30101

(57) ABSTRACT

A wall thickness estimation method includes: obtaining behavioral information that is based on a video in which an organ wall or a blood vessel wall is captured using four-dimensional angiography, the behavioral information being numerical information about changes over time in a position of each of a plurality of predetermined points in the organ wall or the blood vessel wall; generating estimation information using a model trained to take as an input an image indicating a physical parameter based on the behavioral information obtained in the obtaining and output an index indicating a thickness at each of the plurality of predetermined points in the organ wall or the blood vessel wall, the estimation information being information visualizing the thickness; and outputting the estimation information generated in the generating.

### TECHNICAL FIELD

The present invention relates to a wall thickness estimation method and the like. The method is for estimating a thickness of an organ wall or a thickness of a blood vessel wall.

### BACKGROUND ART

A cerebral aneurysm, which is one example of a vascular disease, is an extremely high-risk disease with a fatality rate of more than 50% once the aneurysm ruptures, and is also a socially significant disease due to its high rate of aftereffects. For this reason, preventative treatment (preemptive medicine) to prevent the rupture of cerebral aneurysms is very important, and proper therapeutic intervention is essential.

For proper treatment, it is useful to know information about the wall of the cerebral aneurysm (e.g., the thickness of the wall). This is because it is known that a cerebral aneurysm is more likely to rupture in areas with thin walls than in areas with thick walls. However, even within a single aneurysm, the geometry, such as the thickness, of the aneurysm wall varies from aneurysm to aneurysm.

It is therefore difficult even for experts to infer information about the geometry, such as the thickness, of the aneurysm wall only from the shape of the lumen or the like of the aneurysm wall obtained by computed tomography (CT), magnetic resonance imaging (MRI), and magnetic resonance angiography (MRA).

For example, one known method of estimating the thickness of the wall of a cerebral aneurysm is imaging or visual inspection through craniotomy performed by a doctor. However, this method is highly invasive, places a heavy burden on the patient, and is not a method by which the thickness of the wall of a cerebral aneurysm can be easily estimated.

One example of a known minimally invasive method of measuring the thickness of a blood vessel wall, such as the wall of a cerebral aneurysm, is the ultrasonic diagnostic apparatus disclosed in Patent Literature (PTL) 1. PTL 1 discloses an ultrasonic diagnostic apparatus that generates image data using ultrasonic signals and displays information about the thickness of a blood vessel wall of a subject based on the image data.

### CITATION LIST

### Patent Literature

[PTL 1] Japanese Unexamined Patent Application Publication No. 2013-118932

### SUMMARY OF INVENTION

### Technical Problem

However, the image data obtained using the conventional technique disclosed in PTL 1 is less precise, and therefore, it is difficult to obtain highly accurate information about the blood vessel wall. Furthermore, it is difficult to obtain highly accurate information about not only the blood vessel wall but also an organ wall in a human body and propose information for providing specific treatments for organ diseases or vascular diseases according to the conventional technique.

In view of this, an object of the present invention is to provide a wall thickness estimation method and the like that can generate highly accurate information about an organ wall or a blood vessel wall using a minimally invasive method, thereby proposing useful information for applying specific treatments for organ or vascular diseases.

### Solution to Problem

A wall thickness estimation method according to one aspect of the present invention includes: obtaining behavioral information that is based on a video in which an organ wall or a blood vessel wall is captured using four-dimensional angiography, the behavioral information being numerical information about changes over time in a position of each of a plurality of predetermined points in the organ wall or the blood vessel wall; generating estimation information using a model trained to take as an input an image indicating a physical parameter based on the behavioral information obtained in the obtaining and output an index indicating a thickness at each of the plurality of predetermined points in the organ wall or the blood vessel wall, the estimation information being information visualizing the thickness; and outputting the estimation information generated in the generating.

A computer program according to one aspect of the present invention causes a computer to execute the above-described wall thickness estimation method.

A training method according to one aspect of the present invention includes: obtaining behavioral information that is based on a video in which an organ wall or a blood vessel wall is captured, the behavioral information being numerical information about changes over time in a position of each of a plurality of predetermined points in the organ wall or the blood vessel wall; and training a model using, as training data, one or more datasets constituted by a combination of (i) an image indicating a physical parameter based on the behavioral information at each predetermined point among the plurality of predetermined points, the behavioral information being the behavioral information obtained in the obtaining, and (ii) an index indicating a thickness at the predetermined point among the plurality of predetermined points.

A model construction method according to one aspect of the present invention includes: obtaining the estimation information generated in the above-described generating; and constructing a blood vessel model including the above-described blood vessel wall, the blood vessel model being constructed based on the thickness visualized by the estimation information obtained in the obtaining of the estimation information to cause the blood vessel wall included in the blood vessel model to exhibit a different form according to the thickness.

A wall thickness estimation device according to one aspect of the present invention includes: an obtainer that obtains behavioral information that is based on a video in which an organ wall or a blood vessel wall is captured using four-dimensional angiography, the behavioral information being numerical information about changes over time in a position of each of a plurality of predetermined points in the organ wall or the blood vessel wall; a generator that generates estimation information using a model trained to take as an input an image indicating a physical parameter based on the behavioral information obtained by the obtainer and output an index indicating a thickness at each of the plurality of predetermined points in the organ wall or the blood vessel wall, the estimation information being information visualizing the thickness; and an outputter that outputs the estimation information generated by the generator.

A wall thickness estimation system according to one aspect of the present invention includes: the above-described wall thickness estimation device; a video information processing device that obtains the video, generates the behavioral information, and outputs the behavioral information to the obtainer; and a display that displays the estimation information output by the outputter.

### Advantageous Effects of Invention

According to the wall thickness estimation method and the like of the present invention, it is possible to generate highly accurate information about an organ wall or a blood vessel wall using a minimally invasive method, thereby proposing useful information for applying specific treatments for organ or vascular diseases.

### BRIEF DESCRIPTION OF DRAWINGS

FIG. 1 is a diagram illustrating the configuration of a wall thickness estimation system according to an embodiment.

FIG. 2 is a block diagram illustrating the characteristic functional configuration of a wall thickness estimation device according to the embodiment.

FIG. 3 is a perspective view of a cerebral aneurysm according to the embodiment.

FIG. 4 is a cross-sectional view of a cerebral aneurysm according to the embodiment taken at line IV-IV in FIG. 3.

FIG. 5 is a cross-sectional view of the cerebral aneurysm according to the embodiment taken at line V-V in FIG. 4.

FIG. 6 is a flowchart illustrating a processing sequence in which the wall thickness estimation device according to the embodiment trains a machine learning model.

FIG. 7 is an explanatory diagram illustrating training data according to the embodiment.

FIG. 8 is a flowchart illustrating a processing sequence in which the wall thickness estimation device according to the embodiment estimates the thickness of a wall of a cerebral aneurysm.

FIG. 9 is a diagram indicating an example of estimation information according to the embodiment.

FIG. 10A is a diagram illustrating a still image of a cerebral aneurysm according to the embodiment.

FIG. 10B is a block diagram illustrating the characteristic functional configuration of a model construction system according to Variation 1.

FIG. 10C is a flowchart illustrating a processing sequence by which the model construction system according to Variation 1 constructs a blood vessel model.

FIG. 10D is a schematic diagram illustrating an example of estimation information according to Variation 1.

FIG. 10E is a blood vessel model including a blood vessel wall (aneurysm wall) according to Variation 1.

FIG. 10F is an overall blood vessel model of a brain according to Variation 1.

FIG. 10G is a brain model according to Variation 1.

FIG. 10H is a skull model according to Variation 1.

FIG. 10I is a blood vessel model including a blood vessel wall (aneurysm wall) of an other subject aside from a given subject.

FIG. 11 is a block diagram illustrating the characteristic functional configuration of a wall thickness estimation system according to Variation 2.

FIG. 12 is a flowchart illustrating a processing sequence by which a training device according to Variation 2 trains a machine learning model.

FIG. 13 illustrates one still image (one frame) included in a two-dimensional video according to Variation 2 and an image indicating a depth estimated for the one still image.

### DESCRIPTION OF EMBODIMENTS

An embodiment will be described hereinafter with reference to the drawings. The following embodiment will describe a general or specific example. The numerical values, shapes, materials, elements, the arrangement and connection of the elements, steps, the order of the steps, and the like presented in the following embodiment are merely examples, and do not limit the scope of the present invention. Additionally, of the constituent elements in the following embodiment, constituent elements not denoted in the independent claims will be described as optional constituent elements.

Note also that the drawings are schematic diagrams, and are not necessarily exact illustrations. Configurations that are substantially the same are given the same reference signs in the drawings, and redundant descriptions may be omitted or simplified.

### Embodiment

### [Configuration of Wall Thickness Estimation System]

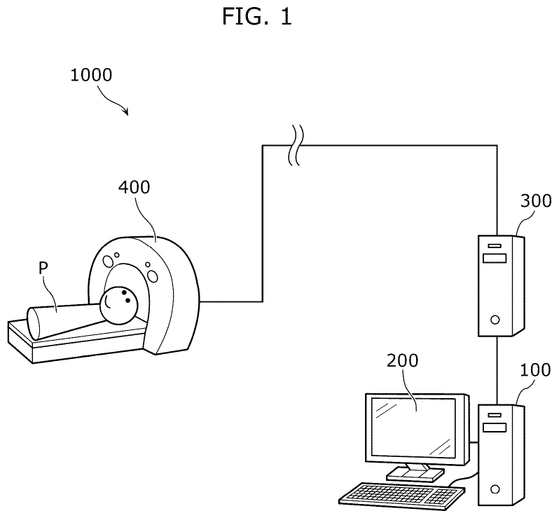

First, the configuration of wall thickness estimation system 1000 according to the present embodiment will be described. FIG. 1 is a diagram illustrating the configuration of wall thickness estimation system 1000 according to the present embodiment.

Wall thickness estimation system 1000 is a system that uses four-dimensional angiography to obtain behavioral information, which is numerical information about changes over time in the position of each of predetermined points, from a video in which an organ wall or a blood vessel wall of subject P is captured. Wall thickness estimation system 1000 further generates estimation information for estimating the thickness of the organ wall or the thickness of the blood vessel wall based on the behavioral information obtained. For example, wall thickness estimation system 1000 estimates a thickness of a cerebral aneurysm, which is one example of a blood vessel wall, in subject P.

Four-dimensional angiography is a technique that adds a time axis to three-dimensional angiography. Three-dimensional angiography is a technique that collects three-dimensional data on blood vessels using an X-ray CT device, an MRI device, or the like and extracts vascular information. Four-dimensional angiography using an X-ray CT device is also referred to as four-dimensional computed tomography angiography (4DCTA).

A video is obtained through the four-dimensional angiography. The video is a time series of three or more still images, and may be, for example, a video obtained over n pulses of the heart (n is a natural number). For example, the video may be a video within a predetermined time period. For example, the predetermined time period may be m seconds (m is a natural number).

Here, an organ wall is a wall of an organ, and organs include chest organs and abdominal organs. For example, chest organs include the heart, lungs, and the like, and abdominal organs include the stomach, intestines, a liver, kidneys, pancreas, and the like, but the examples of chest organs and abdominal organs are not limited thereto. In addition, organs may include chest organs each having a lumen and abdominal organs each having a lumen.

The organ wall is, for example, a wall that divides the organ and other organs. As one example, when the organ is the heart, the organ wall is a wall defined by muscles (myocardium) that divides the heart and the other organs. The organ wall is, for example, a wall that divides regions in the organ. As one example, when the organ is the heart, the organ wall is the interventricular septum that separates the left ventricle and the right ventricle, which are examples of regions in the heart.

The blood vessel wall may be a wall of the blood vessel that is an artery or a vein and may be the wall of an aneurysm or a wall of a varicose vein. For example, the blood vessel wall may be the wall of a cerebral aneurysm, an aortic aneurysm, or visceral aneurysm.

As illustrated in FIG. 1, wall thickness estimation system 1000 includes wall thickness estimation device 100, display 200, video information processing device 300, and video capturing device 400.

Video capturing device 400 is a device that generates a video in which an organ wall or a blood vessel wall is captured using four-dimensional angiography. Video capturing device 400 is, for example, an X-ray CT device or an MRI device. In the present embodiment, video capturing device 400 is an X-ray CT device, and video capturing device 400 includes an X-ray tube that emits X-rays, a detector that receives signals, and a computer.

The detector is located opposite the X-ray tube and detects the X-rays after they have passed through the body of subject P. Using the fact that the absorption of X-rays differs depending on the part of the body of subject P, the computer generates a video including the organ wall or the blood vessel wall in a specific part of the body of subject P. Note that video capturing device 400 has a function of measuring and obtaining an electrocardiogramplex of subject P.

Unlike techniques such as abdominal surgery, open-heart surgery, and craniotomy, the technique of using an X-ray CT device or an MRI device and four-dimensional angiography does not require an incision or the like that places a large burden on the body of subject P, and is therefore a minimally invasive technique. Moreover, the technique of using the X-ray CT device or the MRI device and four-dimensional angiography can generate highly-precise videos.

Video information processing device 300 obtains a video in which an organ wall or a blood vessel wall is captured using four-dimensional angiography generated by video capturing device 400, and generates behavioral information which is numerical information about changes over time in the position of each of a plurality of predetermined points in the organ wall or the blood vessel wall. In other words, the behavioral information is information based on the video in which the organ wall or the blood vessel wall is captured, obtained using four-dimensional angiography.

Here, the behavioral information is numerical information in which a plurality of pairs of (i) a specific time in the video and (ii) the three-dimensional coordinate position of each of a plurality of predetermined points in the organ wall or the blood vessel wall at that specific time are arranged according to the passage of time in which the heart pulsates one time in the video. Note that the plurality of predetermined points means an extremely small region.

Video information processing device 300 outputs the behavioral information to wall thickness estimation device 100. Video information processing device 300 is, for example, a personal computer, but may also be a server device having high computing performance and which is connected to a network.

Wall thickness estimation device 100 obtains the behavioral information generated by video information processing device 300, generates estimation information for estimating the thickness of the organ wall or the thickness of the blood vessel wall based on the obtained behavioral information, and outputs the generated estimation information to display 200. Wall thickness estimation device 100 is, for example, a personal computer, but may also be a server device having high computing performance and which is connected to a network.

Display 200 displays the estimation information output from wall thickness estimation device 100. Specifically, display 200 is a monitor including, for example, a liquid crystal panel or an organic electroluminescent (EL) panel. A television, a smartphone, or a tablet terminal may be used as display 200.

Wall thickness estimation device 100, display 200, and video information processing device 300 may be connected by wires or wirelessly, as long as those devices can send and receive the behavioral information or the estimation information.

In this manner, video information processing device 300 obtains a video in which an organ wall or a blood vessel wall is captured, and generates behavioral information which is numerical information about changes over time in the position of each of a plurality of predetermined points in the organ wall or the blood vessel wall.

Wall thickness estimation device 100 obtains the behavioral information generated by video information processing device 300, and generates estimation information for estimating the thickness of the organ wall or the blood vessel wall based on the obtained behavioral information. Wall thickness estimation device 100 further outputs the generated estimation information to display 200.

In this manner, wall thickness estimation system 1000 uses video information processing device 300 and video capturing device 400 to obtain a video including an organ wall or a blood vessel wall through a minimally invasive technique. Furthermore, wall thickness estimation system 1000 can generate estimation information for estimating the thickness of the organ wall or the thickness of the blood vessel wall using the behavioral information related to the video. Therefore, wall thickness estimation system 1000 can generate highly accurate information about the wall thickness in the vicinity of each of a plurality of predetermined points in the organ wall or the blood vessel wall.

Next, the functional configuration of wall thickness estimation device 100 according to the present embodiment will be described in detail.

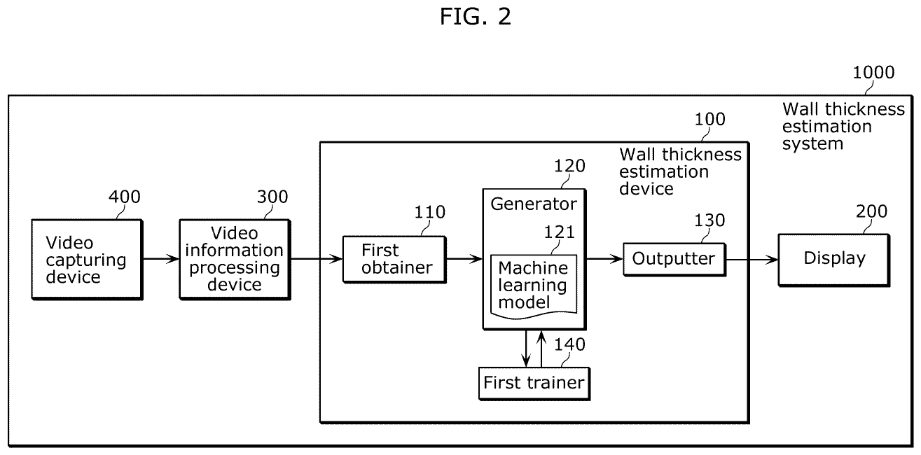

FIG. 2 is a block diagram illustrating the characteristic functional configuration of wall thickness estimation device 100 according to the present embodiment. Wall thickness estimation device 100 includes first obtainer 110 serving as an obtainer, generator 120, outputter 130, and first trainer 140.

First obtainer 110 obtains behavioral information which is numerical information about changes over time in the position of each of a plurality of predetermined points in an organ wall or a blood vessel wall, based on a video in which the organ wall or the blood vessel wall is captured, obtained using four-dimensional angiography. Specifically, first obtainer 110 obtains behavioral information generated by video information processing device 300. First obtainer 110 is, for example, a communication interface for performing wired or wireless communication.

Generator 120 generates estimation information for estimating the thickness of the organ wall or the blood vessel wall based on the behavioral information obtained by first obtainer 110. More specifically, generator 120 has a trained model (here, machine learning model 121), and uses this model to generate estimation information in which the thickness at each of a plurality of predetermined points in an organ wall or a blood vessel wall is visualized.

The trained model is a model in which an image indicating physical parameters based on behavioral information obtained by first obtainer 110 is taken as an input, and an index indicating the thickness at each of a plurality of predetermined points in the organ wall or the blood vessel wall is output. In the present embodiment, first trainer 140 trains the model. Note that in the following, the stated image for generating estimation information may be referred to as a first input image.

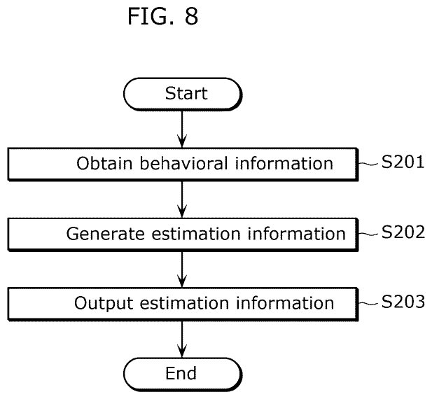

The estimation information is, for example, image information visualizing the thickness at each of the plurality of predetermined points. Note that a method of generating the estimation information will be described later with reference to FIG. 8. Generator 120 is specifically implemented as a processor, a microcomputer, or a dedicated circuit that executes a program.

Outputter 130 outputs the estimation information generated by generator 120. Outputter 130 may output the estimation information generated by generator 120 to display 200. Outputter 130 is, for example, a communication interface for performing wired or wireless communication.

First trainer 140 trains the model using training data. First trainer 140 is specifically implemented as a processor, a microcomputer, or a dedicated circuit that executes a program.

First trainer 140 trains and builds the model. First trainer 140 provides the model built to generator 120. Note that first trainer 140 is not a required element and need not be provided in wall thickness estimation device 100.

The model is a model for generating estimation information.

In the present embodiment, the model is a model built through machine learning using one or more datasets as the training data. One dataset is constituted by a combination of a plurality of (i) an image indicating physical parameters based on the behavioral information from a predetermined point among the plurality of predetermined points in an organ wall or a blood vessel wall and (ii) an index indicating the thickness at the predetermined point.

In other words, the model is a recognition model constructed through machine learning using, as training data, one or more datasets, where each of the one or more datasets is (i) an image indicating physical parameters of the predetermined point and (ii) an index indicating the thickness at the predetermined point.

More specifically, the model is a recognition model built through machine learning, using, as input data, an image indicating physical parameters belonging to each of the one or more datasets serving as the training data, and outputting an index indicating the thickness at the predetermined point belonging to the dataset as output data.

First trainer 140 trains the model using machine learning as described above, as one example. Accordingly, in the present embodiment, the model is machine learning model 121.

First trainer 140 may train the model, for example, using a neural network, and more specifically, using a convolutional neural network (CNN). When the model is a convolutional neural network model, first trainer 140 determines coefficients (weights) of filters in convolutional layers and the like through machine learning based on training data.

First trainer 140 may train the model using an algorithm which is not a neural network.

Note that in the following, the stated image included in the training data may be referred to as a second input image.

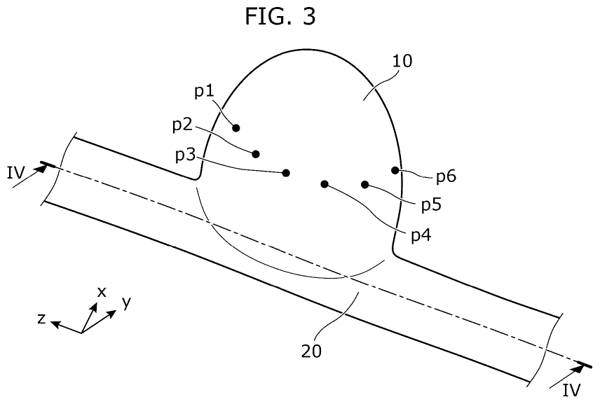

Next, the plurality of predetermined points in information about the behavioral information will described with reference to FIGS. 3 to 5. Although the present embodiment describes a blood vessel wall, the same descriptions apply to organ walls. Here, the blood vessel wall is aneurysm wall 11 of cerebral aneurysm 10.

In FIGS. 3 to 5, the x-axis positive direction is the direction in which cerebral aneurysm 10 extends from parent blood vessel 20, the z-axis is the direction in which parent blood vessel 20 extends, and the y-axis is the direction extending orthogonally to the x- and z-axes, for example. FIGS. 3 to 5, which illustrate parent blood vessel 20, cerebral aneurysm 10, aneurysm wall 11, and a plurality of predetermined points, are general schematic diagrams that can be used to describe not only the brain of subject P, but also the brains of other subjects.

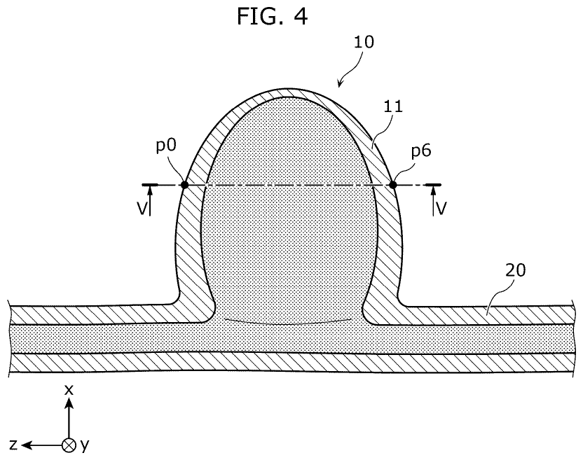

FIG. 3 is a perspective view of cerebral aneurysm 10 according to the present embodiment. FIG. 4 is a cross-sectional view of cerebral aneurysm 10 according to the present embodiment taken at line IV-IV in FIG. 3. Parent blood vessel 20 is one blood vessel among the arteries in the brain. Cerebral aneurysm 10 is an aneurysm in which a portion of parent blood vessel 20 has bulged, extending in the x-axis direction from parent blood vessel 20.

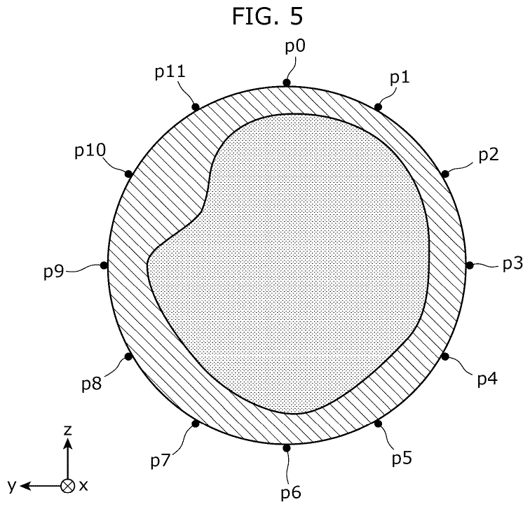

FIG. 5 is a cross-sectional view of cerebral aneurysm 10 according to the present embodiment taken at line V-V in FIG. 4.

As FIG. 5 illustrates, in the cross-sectional view of cerebral aneurysm 10, a plurality of predetermined points are provided in the 0 o'clock direction to the 11 o'clock direction, respectively, so as to correspond to the hours on a clock face. Point p0 is provided in the 0 o'clock direction, and points p1 to P11 are provided in the respective 1 o'clock direction to 11 o'clock direction. In other words, twelve predetermined points are provided at the outer periphery of cerebral aneurysm 10 in the cross-sectional view of cerebral aneurysm 10.

Note that the number of predetermined points is not limited thereto, and for example, 10 to 1000 predetermined points may be provided at the outer periphery of cerebral aneurysm 10 in a single cross-sectional view thereof. Alternatively, although one cross-sectional view is used in the present embodiment, the number of cross-sectional views is not limited thereto. A plurality of cross-sectional views (e.g., 10 to 1000 cross sections) may be used.

Furthermore, for example, 10 to 1,000 predetermined points may be provided at the outer periphery of cerebral aneurysm 10 in each of the cross-sectional views of cerebral aneurysm 10. In this case, 30,000 to 300,000 predetermined points are provided for the one cerebral aneurysm 10.

In addition, the plurality of predetermined points in the blood vessel wall are not limited to the points described above, and can be selected from two or more points in the blood vessel wall. Note that the number of predetermined points is not limited to the number selected from 30,000 to 300,000, and that a number smaller than 30,000 or a number larger than 300,000 may be selected.

The plurality of predetermined points in the blood vessel wall (aneurysm wall 11) in the present embodiment are point p0 to point p11 as described above. In other words, the total number of the plurality of predetermined points present in aneurysm wall 11 is 12.

At each of these twelve predetermined points, first obtainer 110 obtains behavioral information, which is numerical information about changes over time in position. Based on this behavioral information, generator 120 generates estimation information for estimating the thickness of aneurysm wall 11 in the vicinity of each of the predetermined points.

In the present embodiment, the behavioral information is numerical information about changes over time in position during a certain period of time. The certain period of time is, for example, the duration of one pulsation of the heart. Furthermore, the duration of one pulsation of the heart is divided evenly into 100 steps, for example.

Here, the point in time when the pulsation starts is a 0th step, and the point in time when the pulsation ends is a 100th step. The duration of one pulsation of the heart is not limited thereto, and is selected as desired.

Accordingly, the behavioral information includes information about the x-, y-, and z-axis positions of each of the twelve predetermined points at the respective 0th to 100th steps. In other words, the behavioral information is data which is a set of a point of time and coordinate positions (the x-, y-, and z-axis positions) at the point of time for each of the twelve predetermined points. Stated differently, the behavioral information includes time evolution data.

The certain period of time may be a specific number of seconds, e.g., one second, five seconds, or ten seconds. The certain period of time may be subdivided in any manner as long as it is three or more divisions. For example, unlike the above example, the certain period of time may be divided by a number of steps other than 100. Furthermore, the certain period of time need not be divided evenly.

Note that the duration of one pulsation of the heart may be divided evenly into any desired number of steps selected from 10 to 1,000,000 steps, for example. The number of steps is not limited to a number selected from 10 to 1,000,000 steps, and a number smaller than 10 or a number larger than 1,000,000 may be selected.

### [Processing Sequence in Wall Thickness Estimation Method]

Next, a specific processing sequence in the wall thickness estimation method executed by wall thickness estimation device 100 will be described. Although the descriptions will be given referring to a blood vessel wall, the same descriptions also apply to organ walls.

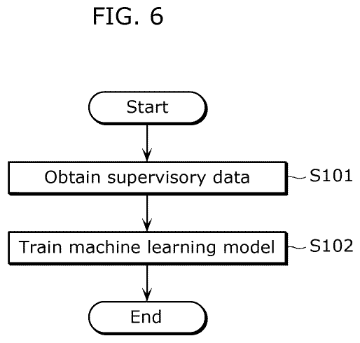

FIG. 6 is a flowchart illustrating a processing sequence in which wall thickness estimation device 100 according to the present embodiment trains a model (machine learning model 121). Note that the processing sequence indicated in the flowchart illustrated in FIG. 6 has been performed before estimating the thickness of aneurysm wall 11 of cerebral aneurysm 10 in subject P.

Here, first trainer 140 trains machine learning model 121 using training data obtained from one or more other subjects other than subject P. Accordingly, for the one or more other subjects, video in which a blood vessel wall is captured is generated by video capturing device 400 using four-dimensional angiography, and wall thickness estimation device 100 obtains behavioral information based on the video. For the sake of simplicity, one other subject B will mainly be used, as an example.

First, first trainer 140 obtains training data for training the model (machine learning model 121) (step S101). The training data may be generated by generator 120, but is not limited thereto, and may be generated by another processor or device.

The training data will be described hereinafter.

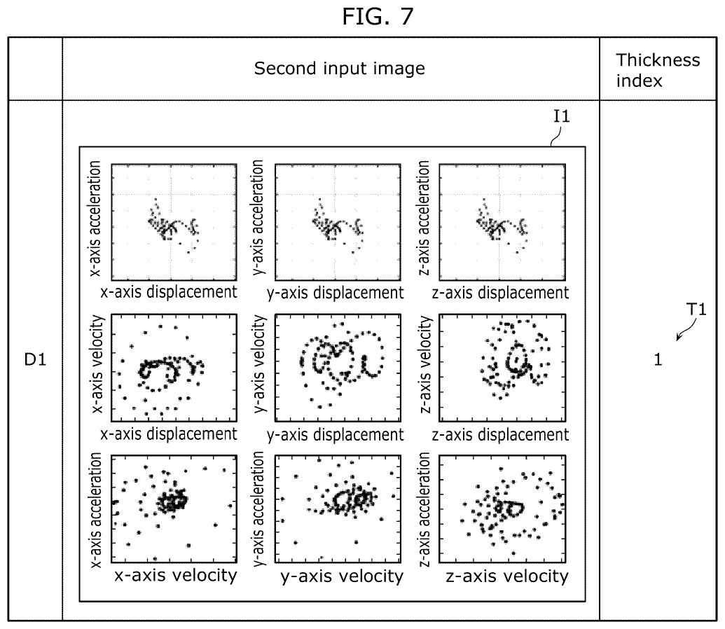

FIG. 7 is an explanatory diagram illustrating the training data according to the present embodiment. The training data is one or more datasets. As an example of the training data, FIG. 7 illustrates one dataset (here, dataset D1) included in the training data.

One dataset is constituted by a combination of (i) an image indicating physical parameters based on the behavioral information at a predetermined point among the plurality of predetermined points (i.e., the second input image) and (ii) an index indicating the thickness at the predetermined point (i.e., a thickness index). As an example, dataset D1 illustrated in FIG. 7 is constituted by second input image I1 and thickness index T1.

In other words, the dataset is data in which the second input image and the thickness index at the predetermined point are one set. The training data is one or more datasets, e.g., preferably at least 100 and at most 1,000,000 datasets, more preferably, at least 1,000 and at most 1,000,000 datasets, and even more preferably, at least 10,000 and at most 1,000,000 datasets. Note that the greater the number of datasets constituting the training data, the better.

For example, the predetermined point corresponding to one dataset is point p0 indicated in FIGS. 3 to 5 for one other subject B. Additionally, the predetermined point corresponding to one other dataset is point p1 indicated in FIGS. 3 to 5 for the one other subject B.

It is desirable to obtain at least 100 and at most 150,000 datasets from the one other subject B, for example. Furthermore, if there are a plurality of other subjects B, it is desirable to obtain at least 100 and at most 150,000 datasets from each of the plurality of other subjects. Note that the number of datasets obtained from the one other subject B may be less than 100, or may be greater than 150,000.

The second input image, which is an image indicating physical parameters based on the behavioral information at the predetermined point, will be described here.

It is desirable that the physical parameters be, for example, parameters about changes over time in the displacement of each of the plurality of predetermined points. In other words, it is desirable that the physical parameters be values calculated from changes over time in the displacement of each of the plurality of predetermined points.

More specifically, the physical parameters are changes over time in the displacement, changes over time in velocity, changes over time in acceleration, changes over time in strain, and the like of the plurality of predetermined points. Note that the displacement is the amount of change in the position at each of steps, taking the position at the 0th step as 0 (the origin), which is the time at which the pulsation starts.

The strain is calculated using the data of points of time and positions included in the behavioral information. The strain calculation method is not particularly limited, and a publicly-known method is used. For example, the strain for one point may be calculated by taking two points, namely the one point (e.g., point p1) and one other point (e.g., point p2) adjacent to the one point, and calculating the strain based on a change in the positions between the two points from a given time (e.g., a tenth step) and the next given time (an 11th step).

The second input image, which is an image indicating physical parameters, will be described further.

It is preferable that the second input image be a two-dimensional image constituted by a graph indicating physical parameters pertaining to one predetermined point among the plurality of predetermined points, and this will be described hereinafter. The second input image is constituted by a plurality of graphs, and in the second input image, the plurality of graphs are arranged in a k×l matrix (k and l are natural numbers). The plurality of graphs may be arranged side by side in a row, or may be arranged side by side in two rows. Here, the second input image is constituted by nine graphs, and the plurality of graphs in the image are arranged in a 3×3 matrix as an example.

The nine graphs are as follows.

The three graphs arranged in the first column are graphs about physical parameters of one predetermined point in the x-axis direction. The three graphs arranged in the second column are graphs about physical parameters of the one predetermined point in the y-axis direction. The three graphs arranged in the third column are graphs about physical parameters of the one predetermined point in the z-axis direction.

The three graphs arranged in the first row are graphs in which the horizontal axis represents the displacement in the respective x-axis, y-axis, and z-axis directions, and the vertical axis represents the acceleration in the respective x-axis, y-axis, and z-axis directions. The three graphs arranged in the second row are graphs in which the horizontal axis represents the displacement in the respective x-axis, y-axis, and z-axis directions, and the vertical axis represents the velocity in the respective x-axis, y-axis, and z-axis directions. The three graphs arranged in the third row are graphs in which the horizontal axis represents the velocity in the respective x-axis, y-axis, and z-axis directions, and the vertical axis represents the acceleration in the respective x-axis, y-axis, and z-axis directions.

Although displacement, velocity, and acceleration are used here for the horizontal and vertical axes of the graph, the graphs are not limited thereto. As described above, one of the parameters about changes over time in the displacement of each of the plurality of predetermined points, such as displacement, velocity, acceleration, or strain, may be used for the horizontal axis, and another may be used for the vertical axis.

The thickness index, which is an index indicating the thickness at a predetermined point among the plurality of predetermined points, will be described next. In FIG. 7, “1”, which is a “numerical value”, is indicated as thickness index T1.

In the present embodiment, the thickness index at the predetermined point is an index based on video (and more specifically, a still image that is part of the video) including a blood vessel wall captured by video capturing device 400, and a tone of cerebral aneurysm 10 in the brain indicated by a surgical image obtained in craniotomy. The thickness index will be described in more detail below.

As described above, in the present embodiment, behavioral information for the other subject B has also been obtained. Likewise, wall thickness estimation device 100 has also obtained a video for the other subject B, which is a video captured of a blood vessel wall by video capturing device 400 realized by an X-ray CT device. Here, one or more still images based on the video obtained by wall thickness estimation device 100 are used. Each of the one or more still images is an image cropped from the video, and is, for example, one frame of the video.

Because video capturing device 400 is an X-ray CT device, each of the one or more still images is one or more CT images. The one or more CT images do not include information indicating a tone for the captured cerebral aneurysm 10 in the brain, and are black and white, or in other words, are achromatic images. In addition, each of the one or more CT images includes information indicating to which positions in the CT image the plurality of predetermined points correspond.

Additionally, as described above, video capturing device 400 may be an MRI device. In this case, each of the one or more still images is one or more MRI images. The one or more MRI images do not contain information indicating a tone for the captured cerebral aneurysm 10 in the brain, and are black and white, or in other words, are achromatic images. In addition, each of the one or more MRI images includes information indicating to which positions in the MRI image the plurality of predetermined points correspond.

Furthermore, a craniotomy is performed on the other subject B.

Wall thickness estimation device 100 obtains a surgical image captured when the craniotomy is performed on the other subject B. The surgical image may be either a two-dimensional or a three-dimensional image, but here, the image is a three-dimensional image. The surgical image includes information indicating a tone for the captured cerebral aneurysm 10 in the brain, and is displayed with chromatic colors.

Wall thickness estimation device 100 then superimposes one of the one or more CT images on the surgical image. Furthermore, wall thickness estimation device 100 determines a region in the surgical image to which each of the plurality of predetermined points in the one CT image corresponds. It should be noted that wall thickness estimation device 100 includes an operation acceptor such as a keyboard, a mouse, a touch panel, or the like, and it is preferable that the stated region be determined by having the operation acceptor accept an operation made by a user of wall thickness estimation device 100. Note that similar processing is performed when video capturing device 400 is an MRI device.

As a result, the predetermined point among the plurality of predetermined points is associated with the region in the surgical image to which the predetermined point corresponds. Furthermore, as described above, the surgical image includes information indicating a tone for the captured cerebral aneurysm 10 in the brain. Accordingly, the predetermined point among the plurality of predetermined points is associated with the information indicating the tone of the region to which the predetermined point corresponds.

A relationship between the tone and the thickness of cerebral aneurysm 10 will be described here.

In general, in cerebral aneurysm 10 in the brain captured in a craniotomy, a region having a weak white tone and a strong red tone corresponds to a region having a weak or thin blood vessel wall. On the other hand, in the captured cerebral aneurysm 10 in the brain, a region having a strong white tone and a weak red tone corresponds to a region having a thick blood vessel wall.

Accordingly, in the present embodiment, when the region in the surgical image corresponding to the predetermined point is a region having a weak white tone and a strong red tone, the thickness index indicating the thickness of the predetermined point is “1”, which is a “numerical value” indicating that the predetermined point is thin. Similarly, when the region in the surgical image corresponding to the predetermined point is a region having a strong white tone and a weak red tone, the thickness index indicating the thickness of the predetermined point is “0”, which is a “numerical value” indicating that the predetermined point is thick.

As a method for determining whether the region in the surgical image is a region having a weak white tone and a strong red tone, or a region having a strong white tone and a weak red tone, it is desirable to use a method in which the pixel values of the region in the surgical image, such as RGB, are determined.

As illustrated in FIG. 7, dataset D1 includes “1” as thickness index T1.

Although 300,000 predetermined points may be obtained from the one other subject B as described above, it is sufficient to obtain at least 100 or more and at most 150,000 datasets from the one other subject B. In other words, it is not necessary to use each of all the obtained predetermined points as training data. Note that as described above, the greater the number of datasets constituting the training data, the better.

As illustrated in FIG. 6, after step S101 is performed, first trainer 140 trains the model using the training data obtained in step S101 (first training step S102). More specifically, first trainer 140 trains machine learning model 121 through machine learning. Furthermore, first trainer 140 outputs the trained machine learning model 121 to generator 120.

FIG. 8 is a flowchart illustrating a processing sequence in which wall thickness estimation device 100 according to the present embodiment estimates the thickness of aneurysm wall 11 of cerebral aneurysm 10.

First obtainer 110 obtains behavioral information through video information processing device 300. The behavioral information is numerical information about changes over time in the position of each of a plurality of predetermined points in aneurysm wall 11 of cerebral aneurysm 10 of subject P (first obtaining step S201).

Next, generator 120 uses the trained machine learning model 121 to generate estimation information in which the thickness at each of the plurality of predetermined points in the blood vessel wall is visualized (generation step S202).

In machine learning model 121, when an image indicating physical parameters based on the behavioral information obtained in first obtaining step S201 (i.e., the first input image) is input, an index indicating the thickness at each of a plurality of predetermined points in the blood vessel wall (i.e., the thickness index) is output.

More specifically, when the first input image pertaining to one point among the plurality of predetermined points is input, the thickness index of that one point is output. Here, a first input image pertaining to each of the plurality of predetermined points in subject P is input, and the thickness index at each of the plurality of predetermined points in subject P is output.

The first input image for generating the estimation information, used in generation step S202, is an image indicating the same physical parameters as the second input image. Accordingly, here, the first input image is as follows.

It is preferable that the first input image be a two-dimensional image constituted by a graph indicating physical parameters pertaining to one predetermined point among the plurality of predetermined points. The first input image is constituted by a plurality of graphs, and in the first input image, the plurality of graphs are arranged in a k×l matrix (k and l are natural numbers). Here, the first input image is constituted by nine graphs, and the plurality of graphs in the image are arranged in a 3×3 matrix.

The nine graphs are as follows.

The three graphs arranged in the first column are graphs about physical parameters of one predetermined point in the x-axis direction. The three graphs arranged in the second column are graphs about physical parameters of the one predetermined point in the y-axis direction. The three graphs arranged in the third column are graphs about physical parameters of the one predetermined point in the z-axis direction.

The three graphs arranged in the first row are graphs in which the horizontal axis represents the displacement in the respective x-axis, y-axis, and z-axis directions, and the vertical axis represents the acceleration in the respective x-axis, y-axis, and z-axis directions. The three graphs arranged in the second row are graphs in which the horizontal axis represents the displacement in the respective x-axis, y-axis, and z-axis directions, and the vertical axis represents the velocity in the respective x-axis, y-axis, and z-axis directions. The three graphs arranged in the third row are graphs in which the horizontal axis represents the velocity in the respective x-axis, y-axis, and z-axis directions, and the vertical axis represents the acceleration in the respective x-axis, y-axis, and z-axis directions.

Like the second input image, displacement, velocity, and acceleration are used for the horizontal and vertical axes of the graphs in the first input image, but the graphs are not limited thereto. As described above, one of the parameters about changes over time in the displacement of each of the plurality of predetermined points, such as displacement, velocity, acceleration, or strain, may be used for the horizontal axis, and another may be used for the vertical axis.

Using this first input image as an input, generator 120 obtains a thickness index at each of the plurality of predetermined points in the blood vessel wall.

At this time, each of the obtained plurality of thickness indices is a “numerical value”, as illustrated in FIG. 7. The higher the “numerical value” of one thickness index is, the lower the thickness at the corresponding predetermined point is, and the lower the “numerical value” of one thickness index is, the higher the thickness at the corresponding predetermined point is. For example, the “numerical value” may be a value of at least 0 and at most 1, but is not limited thereto. It should be noted that when the “numerical value” is a value of at least 0 and at most 1, the closer the “numerical value” of one thickness index is to 1, the lower the thickness at the corresponding predetermined point is, and the closer the “numerical value” of one thickness index is to 0, the higher the thickness at the corresponding predetermined point is.

Generator 120 generates estimation information using the thickness index corresponding to each of the plurality of predetermined points output as described above. Here, the estimation information is, as an example, image information in which the thickness at each of the plurality of predetermined points is visualized, but is not limited thereto. For example, the estimation information may be a table indicating the correspondence between each of the plurality of predetermined points and the thickness index of each of the plurality of predetermined points.

Next, outputter 130 outputs the estimation information generated by generator 120 (outputting step S203). In outputting step S203, outputter 130 transmits, to display 200, image data corresponding to the image information generated by generator 120 in generation step S202, for example.

Display 200 obtains the image data output by outputter 130 and displays an image based on the image data.

Wall thickness estimation device 100 may execute the wall thickness estimation method by reading a computer program recorded on a computer-readable recording medium such as a CD-ROM.

### [Relationship Between Estimation Information and Thickness of Blood Vessel Wall]

A relationship between the estimation information and the thickness of the blood vessel wall will be described next. Here, cerebral aneurysm 10 for subject P will be described with reference to the estimation information obtained in the flowchart illustrated in FIG. 8 and a craniotomy.

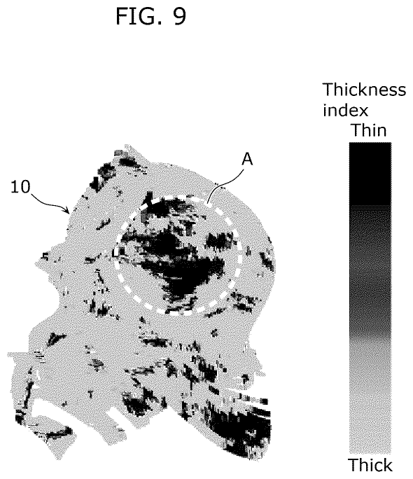

FIG. 9 is a schematic diagram illustrating an example of the estimation information according to the present embodiment.

More specifically, FIG. 9 is an image indicating a schematic diagram of a relationship between (i) the shape of cerebral aneurysm 10 indicated by image information, which is an example of the estimation information output in outputting step S203 in FIG. 8, and (ii) a thickness index at each of a plurality of predetermined points in cerebral aneurysm 10.

In cerebral aneurysm 10 illustrated in FIG. 9, a color indicating the thickness index is applied to the location corresponding to each of the plurality of predetermined points, where a darker color indicates a higher “numerical value” for the thickness index, and a lighter color indicates a lower “numerical value” for the thickness index. Although cerebral aneurysm 10 is represented in black and white in FIG. 9, it should be noted that a schematic diagram of cerebral aneurysm 10 may be represented in full color when outputter 130 actually outputs the schematic diagram.

Furthermore, a craniotomy has been performed for cerebral aneurysm 10 of subject P.

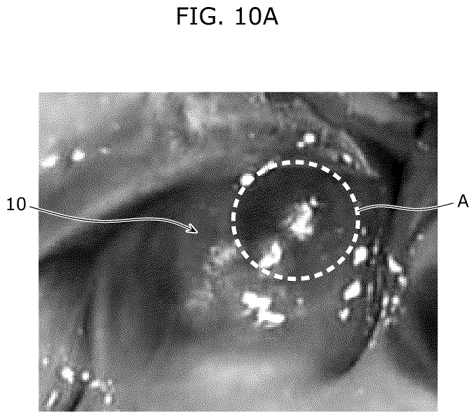

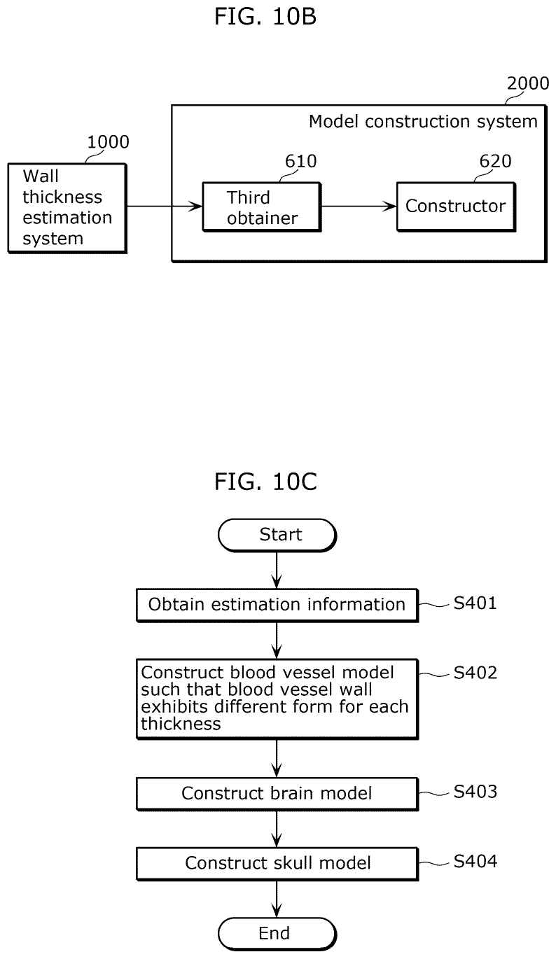

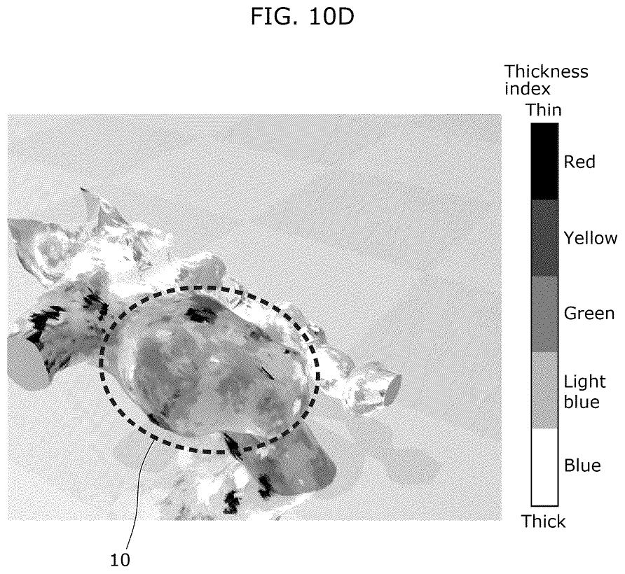

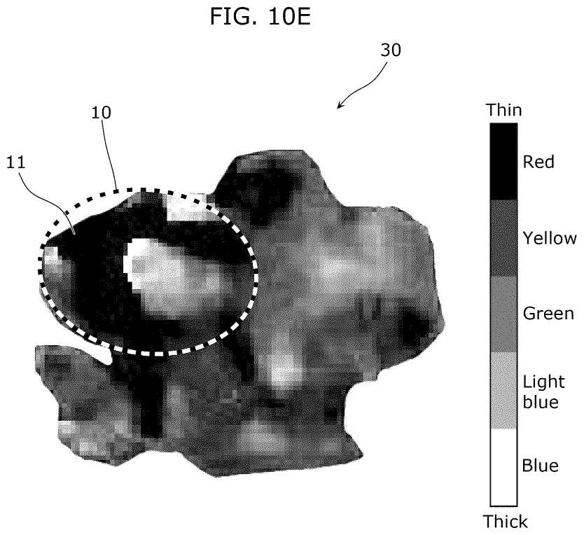

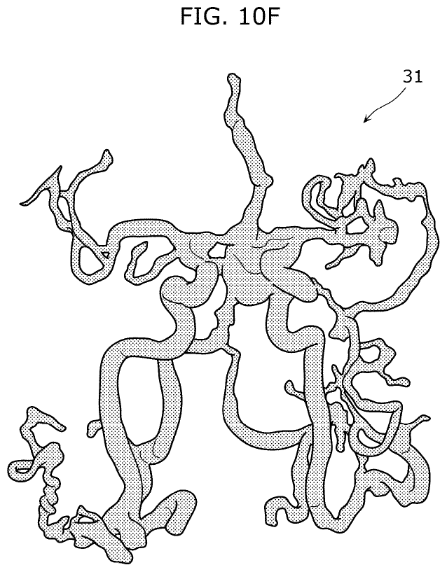

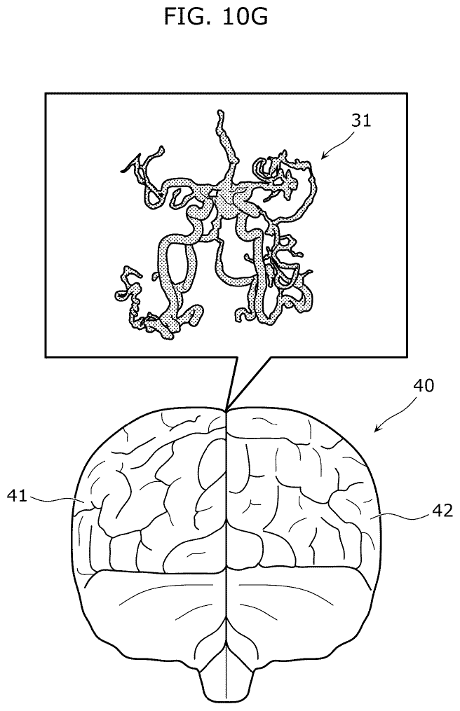

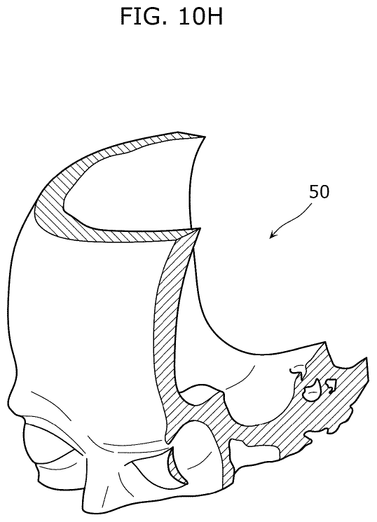

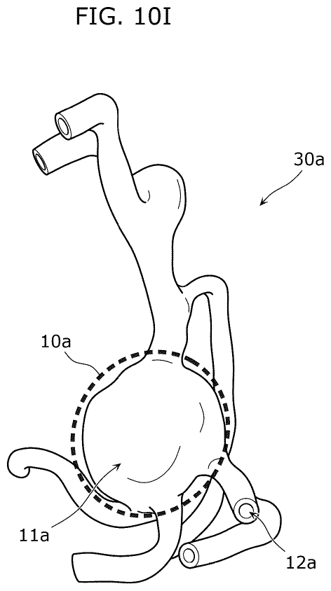

FIG. 10A is a diagram illustrating a still image of cerebral aneurysm 10 according to the present embodiment.

Although cerebral aneurysm 10 is represented in two colors, namely black and white, in FIG. 10A, it should be noted that the still image of cerebral aneurysm 10 is represented in color in an actual craniotomy. For this reason, in FIG. 10A, the region of cerebral aneurysm 10 represented by a dark color is a region having a weak white tone and a strong red tone in the actual craniotomy. In addition, in FIG. 10A, the region of cerebral aneurysm 10 represented by a light color is a region having a strong white tone and a weak red tone in the actual craniotomy.

The shape of the cerebral aneurysm and the region in which the blood vessel wall is thin are revealed by the craniotomy.

Here, the estimation information illustrated in FIG. 9 is compared with the shape of cerebral aneurysm 10 and the tone of the blood vessel wall revealed by the craniotomy illustrated in FIG. 10A.

As illustrated in FIGS. 9 and 10A, cerebral aneurysm 10 illustrated in each image has a similar shape. Furthermore, a circular region A is indicated in each of FIGS. 9 and 10A. Region A in FIG. 9 and region A in FIG. 10A indicate the same region, corresponding to each other.

Inside region A indicated in FIG. 9, there is a region where the color indicating the thickness index is dark, i.e., a region where the “numerical value” indicated by the thickness index is high. Accordingly, the inside of region A indicated in FIG. 9 is estimated to be thin based on the estimation information.

Furthermore, in region A illustrated in FIG. 10A, there is a region where the color is dark, i.e., a region where the white tone is weak and the red tone is strong in the actual craniotomy. Accordingly, the inside of region A indicated in FIG. 10A is estimated to be thin based on the craniotomy. In region A indicated in FIG. 10A, there is a region in which a white color appears due to light when the image is captured, but in the actual cerebral aneurysm 10, in region A, the white tone is weak and the red tone is strong.

In other words, the thickness of cerebral aneurysm 10 estimated based on the estimation information coincides well with the thickness of cerebral aneurysm 10 obtained by the actual craniotomy.

Accordingly, the estimation information illustrated in FIG. 9 can be used as highly accurate information about the thickness of the blood vessel wall.

Such information is information useful for, for example, distinguishing between a cerebral aneurysm which is likely to grow and rupture and a cerebral aneurysm which is unlikely to grow and rupture, and appropriately determining whether treatment is needed.

In other words, the wall thickness estimation method according to the present embodiment makes it possible to propose useful information for providing a specific treatment for a vascular disease by generating highly accurate information about a wall of a blood vessel through a minimally invasive method. Furthermore, the wall thickness estimation method according to the present embodiment is not limited to a blood vessel wall, and can be used to estimate the thickness of an organ wall as well.

In other words, the wall thickness estimation method according to the present embodiment makes it possible to propose useful information for providing a specific treatment for an organ disease by generating highly accurate information about a wall of the organ through a minimally invasive method which does not require abdominal surgery, open-heart surgery, craniotomy, or the like.

### [Variation 1]

The configuration of model construction system 2000 according to Variation 1 on the present embodiment will be described next.

FIG. 10B is a block diagram illustrating the characteristic functional configuration of model construction system 2000 according to the present variation.

Model construction system 2000 is a system for constructing a blood vessel model including a blood vessel wall obtained, for example, using four-dimensional angiography as described above, based on estimation information output from wall thickness estimation system 1000 (and more specifically, outputter 130). Furthermore, if the blood vessel wall included in the blood vessel model is aneurysm wall 11 in cerebral aneurysm 10, model construction system 2000 constructs a brain model into which the blood vessel model is incorporated, and also constructs a skull model containing the brain model.

Before operating on cerebral aneurysm 10 of subject P (i.e., the patient), a doctor explains the operation to subject P. The doctor uses the constructed blood vessel model, brain model, and skull model to explain the operation to subject P. According to the sequence illustrated in FIG. 8, the thickness of aneurysm wall 11 of cerebral aneurysm 10 of subject P is estimated, and a blood vessel model of subject P is constructed based on the estimation information output in outputting step S203. When subject P has the operation explained to him/her, subject P's own blood vessel model is used instead of a general commercially-available model, and subject P can therefore deepen his/her understanding of the operation and undergo the operation with more confidence.

The functional configuration of model construction system 2000 according to the present variation will be described in detail next.

As illustrated in FIG. 10B, model construction system 2000 includes third obtainer 610 and constructor 620.

Third obtainer 610 obtains the estimation information generated in generation step S202. More specifically, third obtainer 610 obtains the estimation information generated in generation step S202 and further output in outputting step S203. Third obtainer 610 is, for example, a communication interface for performing wired or wireless communication.

Constructor 620 constructs a blood vessel model including a blood vessel wall. Constructor 620 constructs the blood vessel model based on a thickness visualized by the estimation information obtained by third obtainer 610. More specifically, constructor 620 constructs the blood vessel model such that a blood vessel wall included in the blood vessel model exhibits a different form for each thickness. Constructor 620 is, for example, a 3D printer.

Next, a specific processing sequence in a model construction method executed by model construction system 2000 will be described. The blood vessel wall included in the blood vessel model will be described here as being aneurysm wall 11 in cerebral aneurysm 10, but the same applies to blood vessel walls other than aneurysm wall 11.

FIG. 10C is a flowchart illustrating a processing sequence by which model construction system 2000 according to the present variation constructs a blood vessel model.

First, third obtainer 610 obtains the estimation information generated in generation step S202 (third obtaining step S401). The estimation information obtained by third obtainer 610 is, for example, image information visualizing the thickness at each of a plurality of predetermined points, but is not limited thereto. For example, the estimation information may be a table indicating the correspondence between each of the plurality of predetermined points and the thickness index of each of the plurality of predetermined points.

FIG. 10D is a schematic diagram illustrating an example of the estimation information according to the present variation. More specifically, like FIG. 9, FIG. 10D is an image indicating a schematic diagram of a relationship between (i) the shape of cerebral aneurysm 10 indicated by image information, which is an example of the estimation information output in outputting step S203 in FIG. 8, and (ii) a thickness index at each of a plurality of predetermined points in cerebral aneurysm 10.

In cerebral aneurysm 10 illustrated in FIG. 10D, a color indicating the thickness index is applied to a location corresponding to each of the plurality of predetermined points, i.e., the blood vessel wall (and more specifically, aneurysm wall 11) has a different color for each thickness. Although illustrated in black and white in FIG. 10D, cerebral aneurysm 10 is actually displayed in red, yellow, green, light blue, and blue, in order from the darkest color. Note, however, that the colors indicating the thickness index are not limited to the five colors of red, yellow, green, light blue, and blue, and may be, for example, red-brown, an intermediate color between red-brown and white, and white, or the like.

In addition, a darker color (a color closer to red) indicates a higher “numerical value” for the thickness index, and a lighter color (a color closer to blue) indicates a lower “numerical value” for the thickness index. As described above, the higher the “numerical value” indicated by the thickness index is, the lower the thickness at the corresponding predetermined point is, and the lower the “numerical value” indicated by the thickness index is, the higher the thickness at the corresponding predetermined point is.

Furthermore, constructor 620 constructs a blood vessel model including a blood vessel wall (aneurysm wall 11) (first construction step S402). Constructor 620 constructs the blood vessel model based on the thickness visualized by the estimation information obtained by third obtainer 610, such that the blood vessel wall included in the blood vessel model has a different form for each thickness.

FIG. 10E is blood vessel model 30 including a blood vessel wall (aneurysm wall 11) according to the present variation. Blood vessel model 30 including the blood vessel wall (aneurysm wall 11) is also blood vessel model 30 including cerebral aneurysm 10. Constructor 620 constructs blood vessel model 30 based on the model diagram (i.e., estimation information) illustrated in FIG. 10D.

Like FIG. 10D, in FIG. 10E, blood vessel model 30 is shown in black and white, but is actually displayed in red, yellow, green, light blue, and blue, in order from the darkest color. In addition, a darker color (a color closer to red) indicates a lower thickness for the blood vessel wall, and a lighter color (a color closer to blue) indicates a higher thickness for the blood vessel wall.

In other words, constructor 620 constructs blood vessel model 30 such that the blood vessel wall included in blood vessel model 30 exhibits a different form for each thickness, and more specifically, such that blood vessel wall (aneurysm wall 11) exhibits a different color for each thickness.

Note that constructor 620 constructs blood vessel model 30 in first construction step S402 through a first construction method or a second construction method, for example, described below.

In the first construction method, constructor 620 constructs blood vessel model 30 by first constructing a model expressing an external shape corresponding to blood vessel model 30, and then coloring or staining the surface of the constructed blood vessel model 30 in red, yellow, green, light blue, and blue. Note, however, that the colors for the surface of the model are not limited to the five colors of red, yellow, green, light blue, and blue, and may be, for example, red-brown, an intermediate color between red-brown and white, and white, or the like.

In this case, for example, constructor 620, which is a 3D printer, constructs the model using a white or transparent material (e.g., filament or UV resin) when constructing the model expressing an external shape corresponding to blood vessel model 30. The model is white, and blood vessel model 30 is constructed by coloring or staining the white model.

In the second construction method, constructor 620 may construct blood vessel model 30 as follows. Constructor 620, which is a 3D printer, constructs blood vessel model 30 using red, yellow, green, light blue, and blue materials (e.g., filaments or UV resins). Constructor 620 is also not limited to the five material colors of red, yellow, green, light blue, and blue, and may use materials that are, for example, red-brown, an intermediate color between red-brown and white, and white. In this case, constructor 620 may construct blood vessel model 30, for example, according to the colors indicated in the model diagram illustrated in FIG. 10D (i.e., the estimation information). According to this construction method, the process of coloring or staining can be omitted.

As illustrated in FIG. 10E, in first construction step S402, blood vessel model 30 is constructed such that the blood vessel wall (aneurysm wall 11) exhibits a different color for each thickness at the plurality of predetermined points. However, the construction is not limited thereto. In first construction step S402, based on the thickness visualized by the estimation information, blood vessel model 30 may be constructed such that the blood vessel wall included in blood vessel model 30 exhibits a different form for each thickness. For example, in first construction step S402, blood vessel model 30 may be constructed such that the blood vessel wall included in blood vessel model 30 has a different tactile feel for each thickness. More specifically, as the thickness increases, the tactile feel of the surface may be made rougher (i.e., the unevenness of the surface may be increased), and as the thickness decreases, the tactile feel of the surface may be made smoother (i.e., the unevenness of the surface may be reduced).

In first construction step S402, constructor 620 constructs blood vessel model 30 including blood vessel wall (aneurysm wall 11), but the construction is not limited thereto. Constructor 620 may also construct a blood vessel model that does not include a blood vessel wall (aneurysm wall 11). In other words, blood vessel model 30 including a blood vessel wall (aneurysm wall 11) constructed in first construction step S402 is a model of a part of a blood vessel in the brain, and thus a blood vessel model not including blood vessel wall (aneurysm wall 11) (i.e., a model of other parts aside from the stated part of the blood vessel in the brain) may also be constructed.

Blood vessel model 30 including the blood vessel wall (aneurysm wall 11) and a blood vessel model not including blood vessel wall (aneurysm wall 11) may be constructed separately by constructor 620, and blood vessel model 30 including the blood vessel wall and the blood vessel model not including the blood vessel wall may then be combined to obtain an overall model of the blood vessels in the brain (called an overall blood vessel model hereinafter). FIG. 10F is overall blood vessel model 31 of a brain according to the present variation.

Note that blood vessel model 30 including the blood vessel wall (aneurysm wall 11) and the blood vessel model not including the blood vessel wall (aneurysm wall 11) each preferably has a magnet. Blood vessel model 30 including the blood vessel wall (aneurysm wall 11) and the blood vessel model not including the blood vessel wall (aneurysm wall 11) may be connected and combined using the magnet in blood vessel model 30 including the blood vessel wall (aneurysm wall 11) and the magnet in the blood vessel model not including the blood vessel wall (aneurysm wall 11). In addition, because two magnets are provided, blood vessel model 30 including the blood vessel wall (aneurysm wall 11) and the blood vessel model not including the blood vessel wall (aneurysm wall 11) can be detached from each other.

Note that the blood vessel model not including the blood vessel wall (aneurysm wall 11) is constructed as follows, as one example. As described above, four-dimensional angiography is a technique that adds a time axis to three-dimensional angiography., and three-dimensional angiography is a technique that collects three-dimensional data on blood vessels using an X-ray CT device, an MRI device, or the like and extracts vascular information. In order to construct the blood vessel model that does not include the blood vessel wall (aneurysm wall 11), for example, in third obtaining step S401, third obtainer 610 obtains the three-dimensional data of the blood vessel from video capturing device 400 or video information processing device 300 of wall thickness estimation system 1000. Next, in first construction step S402, based on the obtained three-dimensional data of the blood vessel, constructor 620 constructs the blood vessel model that does not include the blood vessel wall (aneurysm wall 11).

Note that the obtained three-dimensional data of the blood vessel has information expressing the external shape of the blood vessel, but does not have information expressing the thickness of the blood vessel wall. Therefore, the blood vessel model that does not include the blood vessel wall (aneurysm wall 11) is a model expressing the external shape of the blood vessel, and is not a model expressing the thickness of the blood vessel wall.

Furthermore, constructor 620 constructs a brain model into which blood vessel model 30 constructed in first construction step S402 is to be incorporated (second construction step S403). FIG. 10G is brain model 40 according to the present variation. In the present variation, brain model 40 is a model into which overall blood vessel model 31 of the brain, including blood vessel model 30, is to be incorporated. Brain model 40 includes right brain model 42 and left brain model 41, and is configured such that right brain model 42 and left brain model 41 can be separated from each other. With right brain model 42 and left brain model 41 separated, overall blood vessel model 31 of the brain is placed between right brain model 42 and left brain model 41 and sandwiched between right brain model 42 and left brain model 41 such that overall blood vessel model 31 of the brain is incorporated into brain model 40.

In the present variation, brain model 40 includes right brain model 42 and left brain model 41, but the present variation is not limited thereto. In another example, the brain model includes a right side brain model and a left side brain model, and each of the right side brain model and the left side brain model may include the cerebrum, the midbrain, the cerebellum, and the brainstem.

In second construction step S403, brain model 40 is constructed as follows, for example.

As described above, three-dimensional data on a blood vessel is collected by an X-ray CT device, an MRI device, or the like. When three-dimensional data on the blood vessel is collected, three-dimensional data on the brain and three-dimensional data on the skull are also collected in addition to the three-dimensional data on the blood vessel. In the present variation, for example, in third obtaining step S401, third obtainer 610 also obtains the three-dimensional data of the brain from video capturing device 400 or video information processing device 300 of wall thickness estimation system 1000. After first construction step S402, in second construction step S403, constructor 620 constructs brain model 40 based on the obtained three-dimensional data of the brain.

Next, constructor 620 constructs a skull model for containing brain model 40 constructed in second construction step S403 (third construction step S404). FIG. 10H is skull model 50 according to the present variation. In the present variation, skull model 50 includes overall blood vessel model 31 of the brain, including blood vessel model 30, and brain model 40.

Note that to illustrate the space in which brain model 40 and the like are contained, FIG. 10H illustrates only parts of the model corresponding to a part of the skull, and parts of the model corresponding to other parts of the skull are not illustrated. However, in the actual third construction step S404, a model corresponding to the entire skull (i.e., skull model 50) is constructed.

Skull model 50 also includes the occipital bone, the temporal bone, the parietal bone, the frontal bone, and the sphenoid bone, which make up the cerebral cranium (neurocranium), as well as the ethmoid bone, the lacrimal bone, the nasal bone, the maxillary bone, the mandibular bone, the palatal bone, the inferior turbinate, the cheekbone, the vomer, and the hyoid bone, which make up the facial cranium (visceral cranium).

In third construction step S404, skull model 50 is constructed as follows, for example.

As described above, three-dimensional data on the skull is also collected by the X-ray CT device, the MRI device, or the like. In the present variation, for example, in third obtaining step S401, third obtainer 610 also obtains the three-dimensional data of the skull from video capturing device 400 or video information processing device 300 of wall thickness estimation system 1000. After first construction step S402 and second construction step S403, in third construction step S404, constructor 620 constructs skull model 50 based on the obtained three-dimensional data of the skull.

As described above, in the present variation, subject P's own blood vessel model 30 is constructed based on the estimation information, and further, subject P's own brain model 40 and skull model 50 are constructed.

Additionally, in second construction step S403, brain model 40 may be constructed as follows. For example, three-dimensional data of a brain in a commercially-available brain model of a typical size may be used instead of three-dimensional data of the brain of subject P. Such three-dimensional data of the brain is obtained by measuring a commercially-available brain model of a typical size using an X-ray CT device, an MRI device, a 3D scanner capable of measuring a three-dimensional shape, and the like. In this case, brain model 40 of subject P him/herself is not constructed.

The doctor uses the constructed blood vessel model 30, brain model 40, and skull model 50 of subject P him/herself (i.e., the patient) when explaining the operation for cerebral aneurysm 10 for subject P. In particular, for blood vessel model 30, since the blood vessel wall in blood vessel model 30 exhibits a different form for each thickness, subject P can easily understand which part of the blood vessel wall is thick or which part is thin. Subject P will be able to deepen his/her understanding of the operation and will therefore be able to undergo the operation with more confidence. In other words, the model construction method according to the present variation is a method that can assist a doctor in explaining the operation to subject P (i.e., the patient).

Furthermore, for example, the doctor may rehearse the operation using the constructed blood vessel model 30, brain model 40, and skull model 50 prior to performing the actual operation. This enables the doctor to perform the operation with confidence and peace of mind. In other words, the model construction method according to the present variation is a method that can assist a doctor in performing the operation with confidence and peace of mind.

Furthermore, in the present variation, blood vessel model 30 and brain model 40 constructed in first construction step S402 and second construction step S403 may be flexible and elastic. For example, blood vessel model 30 and brain model 40 may deform when the doctor or subject P touches blood vessel model 30 and brain model 40 with his/her own hand, blood vessel model 30 and brain model 40 may return to their original shape when the doctor or subject P removes his/her hand. For example, blood vessel model 30 and brain model 40 may be constructed using silicone resin or the like as a material in first construction step S402 and second construction step S403.

Furthermore, in the present variation, constructor 620 that constructs brain model 40 and skull model 50 in second construction step S403 and third construction step S404 is a 3D printer, but is not limited thereto. For example, constructor 620 may be molds in which brain model 40 and skull model 50 are molded, and resin may be poured into the molds to construct brain model 40 and skull model 50.

FIG. 10I is blood vessel model 30a including a blood vessel wall (aneurysm wall 11a) of an other subject C aside from subject P. Blood vessel model 30a including the blood vessel wall (aneurysm wall 11a) is also blood vessel model 30a including cerebral aneurysm 10a. Blood vessel model 30a illustrated in FIG. 10I is constructed in the same manner as blood vessel model 30 of subject P.

Additionally, as illustrated in FIG. 10I, blood vessel model 30a is also provided with hole 12a corresponding to a flow channel for blood to flow, and is tubular. Like blood vessel model 30a, blood vessel model 30 illustrated in FIG. 10E is tubular. Note that hole 12a need not be provided, i.e., blood vessel model 30 illustrated in FIG. 10E need not be tubular.

### [Variation 2]

The configuration of wall thickness estimation system 1000a according to Variation 2 on the present embodiment will be described next.

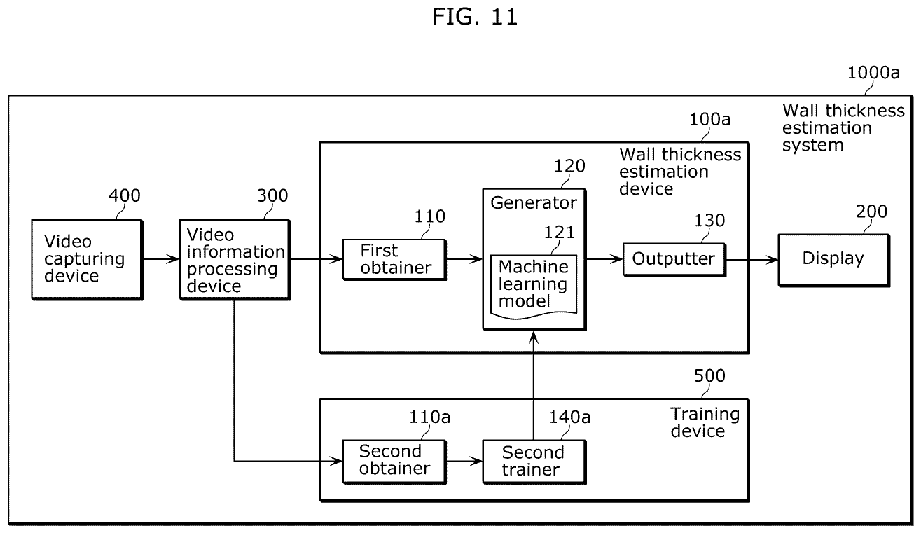

FIG. 11 is a block diagram illustrating the characteristic functional configuration of wall thickness estimation system 1000a according to the present variation.

Wall thickness estimation system 1000a differs from wall thickness estimation system 1000 according to the embodiment primarily in that training device 500 is provided, and that wall thickness estimation device 100a does not include first trainer 140.

Training device 500 obtains behavioral information generated by video information processing device 300. Training device 500 trains a model (here, machine learning model 121) using, as training data, one or more datasets constituted by a combination of an image indicating physical parameters based on the obtained behavioral information and a thickness index. Training device 500 outputs the trained model to generator 120 provided in wall thickness estimation device 100a. Training device 500 is, for example, a personal computer, but may also be a server device having high computing performance and which is connected to a network.

Training device 500 includes second obtainer 110a and second trainer 140a.

Second obtainer 110a obtains behavioral information based on a video in which an organ wall or a blood vessel wall is captured, the behavioral information being numerical information about changes over time in a position of each of a plurality of predetermined points in the organ wall or the blood vessel wall. More specifically, second obtainer 110a obtains behavioral information which is numerical information about changes over time in the position of each of a plurality of predetermined points in an organ wall or a blood vessel wall, based on a video in which the organ wall or the blood vessel wall is captured, obtained using four-dimensional angiography. Specifically, second obtainer 110a obtains behavioral information generated by video information processing device 300. Second obtainer 110a is, for example, a communication interface for performing wired or wireless communication.

Second trainer 140a trains the model using one or more datasets as training data. Second trainer 140a is specifically implemented as, a processor, a microcomputer, or a dedicated circuit that executes a program.

One dataset is constituted by a combination of a plurality of (i) an image indicating physical parameters based on the behavioral information from a predetermined point among the plurality of predetermined points in an organ wall or a blood vessel wall and (ii) an index indicating the thickness at the predetermined point.

Note that the behavioral information is information obtained by second obtainer 110a.

In the present variation, the training data may be data generated by second trainer 140a.

Next, a specific processing sequence in a training method executed by training device 500 will be described. Although the descriptions will be given referring to a blood vessel wall, the same descriptions also apply to organ walls here as well.

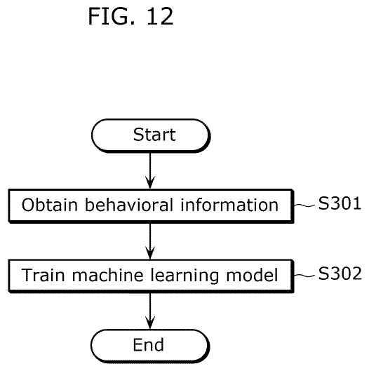

FIG. 12 is a flowchart illustrating a processing sequence by which training device 500 according to present variation trains a model (machine learning model 121).

First, second obtainer 110a obtains behavioral information (second obtaining step S301).

Furthermore, second trainer 140a generates training data and trains the model based on the obtained behavioral information (second training step S302). More specifically, second trainer 140a trains machine learning model 121. Second trainer 140a outputs machine learning model 121 which has been trained to generator 120.

As described in the present variation, wall thickness estimation device 100a, which generates estimation information, and training device 500, which trains the model (machine learning model 121), may be separate devices.

Although the foregoing describes second obtainer 110a as obtaining behavioral information based on a video of an organ wall or a blood vessel wall captured using four-dimensional angiography, the method is not limited thereto. The video may be a video captured using a two-dimensional video capturing device (two-dimensional video). In other words, second obtainer 110a may obtain behavioral information which is numerical information about changes over time in the position of each of a plurality of predetermined points in an organ wall or a blood vessel wall, based on a video in which the organ wall or the blood vessel wall is captured, obtained using a two-dimensional video capturing device (a two-dimensional image).

The two-dimensional video is, for example, a video about one or more other subjects other than subject P. For the sake of simplicity, one other subject D will mainly be used, as an example.

The two-dimensional video is a surgical video captured when abdominal surgery or a craniotomy is performed on the other subject D. Unlike video obtained using four-dimensional angiography, the two-dimensional video is not three-dimensional data, i.e., is data that does not include information indicating a depth of the two-dimensional video. In this case, video capturing device 400 corresponds to a two-dimensional video capturing device (e.g., a camera).

Furthermore, video information processing device 300 obtains the two-dimensional video captured by video capturing device 400. Video information processing device 300 estimates a depth of the two-dimensional video and generates depth information indicating the estimated depth. For example, video information processing device 300 estimates the depth of the two-dimensional video using a depth estimation AI model, but the method is not limited thereto, and other methods may be used.

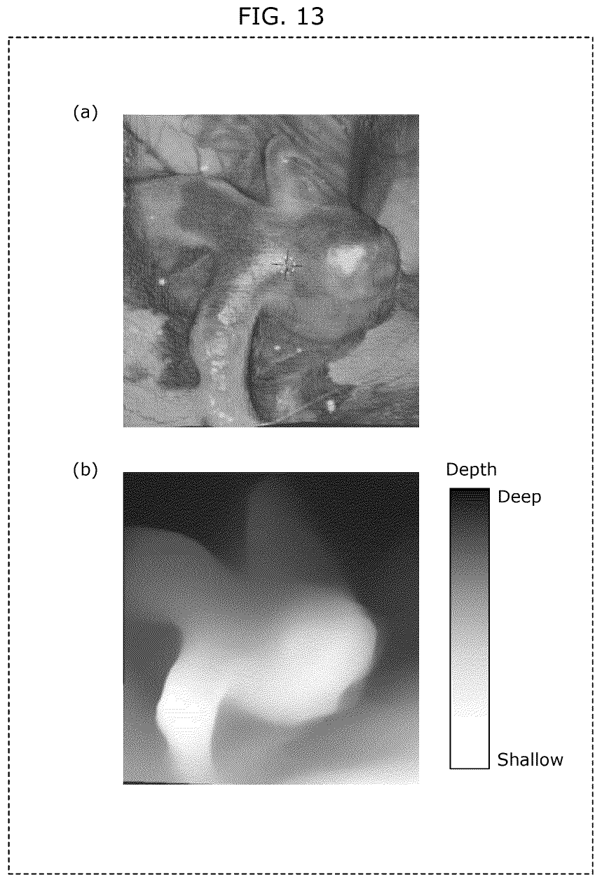

FIG. 13 illustrates one still image (one frame) included in a two-dimensional video according to the present variation and an image indicating a depth estimated for the one still image. More specifically, (a) in FIG. 13 illustrates one still image (one frame) included in the two-dimensional video, and (b) in FIG. 13 illustrates an image indicating a depth estimated for the one still image. In (b) of FIG. 13, a greater depth is indicated by a darker color, and a lower depth is indicated by a lighter color. Like the video obtained using four-dimensional angiography, the information obtained by combining the two-dimensional video and the depth information is three-dimensional data.

Video information processing device 300 generates behavioral information which is numerical information about changes over time in the position of each of a plurality of predetermined points in the organ wall or the blood vessel wall, based on the video in which a blood vessel wall or an organ wall is captured using the two-dimensional video capturing device and the depth information indicating the estimated depth. Second obtainer 110a then obtains the generated behavioral information. In other words, the behavioral information obtained by second obtainer 110a is information based on a video in which an organ wall or a blood vessel wall obtained using a two-dimensional video capturing device.

In this manner, even if the video is a video obtained using a two-dimensional video capturing device (a two-dimensional video), the behavioral information is estimated and obtained by second obtainer 110a in the same manner as when the video is a video obtained using four-dimensional angiography, as described above. Second training step S302, which is a subsequent process, is performed in the same manner.

### [Effects, etc.]

As described above, a wall thickness estimation method according to the present embodiment includes first obtaining step S201, generation step S202, and outputting step S203. In first obtaining step S201, behavioral information which is numerical information about changes over time in the position of each of a plurality of predetermined points in an organ wall or a blood vessel wall is obtained, based on a video in which the organ wall or the blood vessel wall is captured, obtained using four-dimensional angiography. In generation step S202, estimation information is generated using a model trained to take as an input an image indicating a physical parameter based on the behavioral information obtained in first obtaining step S201 and output an index indicating a thickness at each of the plurality of predetermined points in the organ wall or the blood vessel wall, the estimation information being information visualizing the thickness. In outputting step S203, the estimation information generated in generation step S202 is output.

In this manner, in the wall thickness estimation method, for example, a video in which the blood vessel wall is captured is generated using an X-ray CT device or an MRI device, and four-dimensional angiography. For example, the video in which the blood vessel wall is captured is obtained using a minimally invasive method compared with methods such as craniotomy. The wall thickness information method makes it possible to generate estimation information which is information that visualizes the thickness at each of the plurality of predetermined points in the blood vessel wall, using the behavioral information related to the video. The thickness of the blood vessel wall estimated based on the estimation information coincides well with the thickness of the blood vessel wall obtained by craniotomy.

In other words, the wall thickness estimation method can generate highly accurate information about the wall thickness in the vicinity of each of the plurality of predetermined points in the blood vessel wall. In the present embodiment, for example, the thickness of aneurysm wall 11 of cerebral aneurysm 10 is estimated. Such information is information useful for, for example, distinguishing between a cerebral aneurysm which is likely to grow and rupture and a cerebral aneurysm which is unlikely to grow and rupture, and appropriately determining whether treatment is needed.

Note that the wall thickness estimation method is not limited to the thickness of a blood vessel wall, and can be used to estimate the thickness of an organ wall.

In other words, the wall thickness estimation method according to the present embodiment makes it possible to generate highly accurate information about an organ wall or a blood vessel wall using a minimally invasive method, thereby proposing useful information for applying specific treatments for organ or vascular diseases.

Additionally, the wall thickness estimation method according to the present embodiment includes first training step S102. In first training step S102, the model is trained using one or more datasets as training data, each of the datasets being constituted by a combination of (i) the image indicating the physical parameter based on the behavioral information at each predetermined point among the plurality of predetermined points and (ii) the index indicating the thickness at the predetermined point.

Through this, the model can output an index indicating the thickness based on an input image indicating a physical parameters. Accordingly, the wall thickness estimation method according to the present embodiment can generate more accurate information about an organ wall or a blood vessel wall using a minimally invasive method.

Additionally, the first training step according to the present embodiment trains the model using machine learning.

Through this, in generation step S202, the estimation information can be generated using a model trained through machine learning (machine learning model 121). Accordingly, the wall thickness estimation method according to the present embodiment can generate more accurate information about an organ wall or a blood vessel wall using a minimally invasive method.

Additionally, the estimation information according to the present embodiment is image information visualizing the thickness.

Through this, the estimation information is obtained as image information. Accordingly, for example, a doctor or the like can visually obtain highly accurate information about the thickness of the organ wall or the blood vessel wall.

Additionally, in the wall thickness estimation method, the blood vessel wall may be a wall of an arterial aneurysm or a varicose vein.

In this manner, the wall thickness estimation method can estimate the thickness of the wall of the arterial aneurysm or the thickness of the wall of the varicose vein.

Additionally, in the wall thickness estimation method according to the present embodiment, the blood vessel wall is aneurysm wall 11 of cerebral aneurysm 10.

In this manner, the wall thickness estimation method can estimate the thickness of aneurysm wall 11 of cerebral aneurysm 10.

Additionally, in the wall thickness estimation method, the blood vessel wall may be a blood vessel wall of an artery or a vein.

In this manner, the wall thickness estimation method can estimate the thickness of the blood vessel wall of an artery or a vein.

Additionally, a computer program may cause a computer to execute the above-described wall thickness estimation method.

Through this, the above-described wall thickness estimation method is executed by a computer.

Additionally, a training method according to Variation 2 includes second obtaining step S301 and second training step S302. In second obtaining step S301, behavioral information that is based on a video in which an organ wall or a blood vessel wall is captured is obtained, the behavioral information being numerical information about changes over time in a position of each of a plurality of predetermined points in the organ wall or the blood vessel wall. In second training step S302, a model is trained using, as training data, one or more datasets constituted by a combination of (i) an image indicating a physical parameter based on the behavioral information at each predetermined point among the plurality of predetermined points, the behavioral information being the behavioral information obtained in the second obtaining step, and (ii) an index indicating a thickness at the predetermined point among the plurality of predetermined points.

When such a model is used in generation step S202 according to the present embodiment, the model can output an index indicating a thickness based on the input image indicating a physical parameter. Accordingly, a wall thickness estimation method using the training method according to Variation 2 can generate more accurate information about an organ wall or a blood vessel wall using a minimally invasive method.

In the training method according to Variation 2, the video is obtained using four-dimensional angiography or a two-dimensional video capturing device.

Through this, second obtaining step S301 can obtain behavioral information based on a video captured through four-dimensional angiography or using a two-dimensional video capturing device.

A model construction method according to Variation 1 includes: third obtaining step S401 in which the estimation information generated in the above-described generation step S202 is obtained; and first construction step S402 in which blood vessel model 30 including the above-described blood vessel wall is constructed, blood vessel model 30 being constructed based on the thickness visualized by the estimation information obtained in third obtaining step S401 to cause the blood vessel wall included in blood vessel model 30 to exhibit a different form according to the thickness.

In Variation 1, blood vessel model 30 of subject P him/herself is constructed. A doctor uses the constructed blood vessel model 30 of subject P him/herself to explain the operation to subject P (i.e., the patient). In particular, for blood vessel model 30, since the blood vessel wall in blood vessel model 30 exhibits a different form for each thickness, subject P can easily understand which part of the blood vessel wall is thick or which part is thin. Accordingly, subject P will be able to deepen his/her understanding of the operation and will therefore be able to undergo the operation with more confidence. In other words, the model construction method according to Variation 1 is a method that can assist a doctor in explaining the operation to subject P (i.e., the patient).

Furthermore, for example, the doctor may rehearse the operation using the constructed blood vessel model 30 prior to performing the actual operation. This enables the doctor to perform the operation with confidence and peace of mind. In other words, the model construction method according to the present variation is a method that can assist a doctor in performing the operation with confidence and peace of mind.

In the model construction method according to Variation 1, in first construction step S402, blood vessel model 30 is constructed such that the blood vessel wall exhibits a different color according to the thickness.

This makes it easier for subject P to understand which part of the blood vessel wall is thick or which part is thin. Subject P will be able to further deepen his/her understanding of the operation and will therefore be able to undergo the operation with more confidence. In other words, a model construction method that can more easily assist a doctor in explaining an operation to subject P (i.e., the patient) is realized.

In the model construction method according to Variation 1, the blood vessel wall included in blood vessel model 30 constructed in first construction step S402 is aneurysm wall 11 of cerebral aneurysm 10. The model construction method according to Variation 1 includes second construction step S403, in which brain model 40 into which blood vessel model 30 constructed in first construction step S402 is to be incorporated.

In Variation 1, blood vessel model 30 and brain model 40 of subject P him/herself are constructed. The doctor uses the constructed blood vessel model 30 and brain model 40 of subject P him/herself (i.e., the patient) when explaining the operation for cerebral aneurysm 10 for subject P. If the blood vessel wall included in blood vessel model 30 is aneurysm wall 11 in cerebral aneurysm 10, subject P can easily understand where cerebral aneurysm 10 and aneurysm wall 11 are located in subject P's brain. Accordingly, subject P will be able to deepen his/her understanding of the operation and will therefore be able to undergo the operation with more confidence. In other words, a model construction method that can more easily assist a doctor in explaining an operation to subject P (i.e., the patient) is realized.

The model construction method according to Variation 1 includes third construction step S404, in which skull model 50 for containing brain model 40 constructed in second construction step S403 is constructed.

In Variation 1, a blood vessel model and a brain model of subject P him/herself are constructed. The doctor uses the constructed blood vessel model 30, brain model 40, and skull model 50 of subject P him/herself (i.e., the patient) when explaining the operation for cerebral aneurysm 10 for subject P. Subject P can easily understand the positional relationships between cerebral aneurysm 10, aneurysm wall 11, the brain, and the skull. Accordingly, subject P will be able to deepen his/her understanding of the operation and will therefore be able to undergo the operation with more confidence. In other words, a model construction method that can more easily assist a doctor in explaining an operation to subject P (i.e., the patient) is realized.

Skull model 50 also includes the occipital bone, the temporal bone, the parietal bone, the frontal bone, and the sphenoid bone, which make up the cerebral cranium (neurocranium), as well as the ethmoid bone, the lacrimal bone, the nasal bone, the maxillary bone, the mandibular bone, the palatal bone, the inferior turbinate, the cheekbone, the vomer, and the hyoid bone, which make up the facial cranium (visceral cranium).

Through this, subject P can easily understand the positional relationship between the frontal region and the occipital region of the head, i.e., the front and rear parts of the face. Accordingly, subject P will be able to deepen his/her understanding of the operation and will therefore be able to undergo the operation with more confidence. In other words, a model construction method that can more easily assist a doctor in explaining an operation to subject P (i.e., the patient) is realized.

Wall thickness estimation device 100 according to the present embodiment includes first obtainer 110, generator 120, and outputter 130. First obtainer 110 obtains behavioral information that is based on a video in which an organ wall or a blood vessel wall is captured using four-dimensional angiography, the behavioral information being numerical information about changes over time in a position of each of a plurality of predetermined points in the organ wall or the blood vessel wall. Generator 120 generates estimation information using a model trained to take as an input an image indicating a physical parameter based on the behavioral information obtained by first obtainer 110 and output an index indicating a thickness at each of the plurality of predetermined points in the organ wall or the blood vessel wall, the estimation information being information visualizing the thickness. Outputter 130 outputs the estimation information generated by generator 120.

In this manner, in wall thickness estimation device 100, for example, a video in which the blood vessel wall is captured is generated using an X-ray CT device or an MRI device, and four-dimensional angiography. For example, the video in which the blood vessel wall is captured is obtained using a minimally invasive method compared with methods such as craniotomy. Wall thickness estimation device 100 makes it possible to generate estimation information which is information that visualizes the thickness at each of the plurality of predetermined points in the blood vessel wall, using the behavioral information related to the video. The thickness of the blood vessel wall estimated based on the estimation information coincides well with the thickness of the blood vessel wall obtained by craniotomy.

In other words, wall thickness estimation device 100 is capable of generating highly accurate information about the wall thickness in the vicinity of each of the plurality of predetermined points in the blood vessel wall. In the present embodiment, for example, the thickness of aneurysm wall 11 of cerebral aneurysm 10 is estimated. Such information is information useful for, for example, distinguishing between a cerebral aneurysm which is likely to grow and rupture and a cerebral aneurysm which is unlikely to grow and rupture, and appropriately determining whether treatment is needed.

Note that wall thickness estimation device 100 is not limited to the thickness of a blood vessel wall, and can be used to estimate the thickness of an organ wall.

In other words, wall thickness estimation device 100 according to the present embodiment makes it possible to propose highly accurate information about an organ wall or a blood vessel wall using a minimally invasive method, thereby providing useful information for applying specific treatments for organ or vascular diseases.

Wall thickness estimation system 1000 according to the present embodiment includes: the above-described wall thickness estimation device 100; video information processing device 300 that obtains the video, generates the behavioral information, and outputs the behavioral information to first obtainer 110; and display 200 that displays the estimation information output by outputter 130.

As described above, wall thickness estimation device 100 according to the present embodiment can generate accurate information about an organ wall or a blood vessel wall using a minimally invasive method. Accordingly, wall thickness estimation system 1000 according to the present embodiment including wall thickness estimation device 100 can propose useful information for applying specific treatments for organ or vascular diseases.

Furthermore, by having the estimation information visualized and displayed, for example, a doctor or the like can obtain highly accurate information about the thickness of the organ wall or the thickness of the blood vessel wall.

### Other Embodiments

Although a wall thickness estimation method and the like according to the embodiment and variations have been described above, the present invention is not limited to the above embodiment.

First trainer 140 may also update the model (machine learning model 121) through machine learning.

The updating of machine learning model 121 by first trainer 140 need not be performed in real time, and may be performed at a later time using the second input image and the thickness index associated with the second input image as the training data.

In addition, the inventors also made the following verifications. In the foregoing embodiment, when first trainer 140 trains the model (machine learning model 121), training data obtained from a plurality of other subjects other than subject P is used. Here, the inventors replaced this training data with training data obtained from subject P and a plurality of other subjects for verification. In other words, first trainer 140 trained machine learning model 121 using training data obtained from subject P and a plurality of other subjects. Even in such a case, generator 120 can use machine learning model 121 to generate the estimation information of subject P.

Note that in the present embodiment and the variations, the thickness index at the predetermined point is an index obtained based on a video including a blood vessel wall captured by video capturing device 400 and a color tone of cerebral aneurysm 10 in the brain indicated by a surgical image obtained in a craniotomy.

However, the obtainment is not limited thereto, and the thickness index at the predetermined point may be obtained based on the video and other information different from the surgical image. The other information is information in which a mass at each of the plurality of predetermined points obtained by mathematical analysis is estimated, for example.

In this case, the higher the mass at each of the predetermined points is, the greater the thickness at the predetermined point is, and the lower the mass at each of the predetermined points is, the lower the thickness at the predetermined point is. Accordingly, the thickness index may be set to “0” for 20000 points from the heaviest among the plurality of predetermined points, and a thickness index of may be set to “1” for 2000 points from the lightest among the plurality of predetermined points. The thickness index may be obtained in this manner.

The foregoing embodiment describes methods of obtaining behavioral information using actual cases and four-dimensional angiography. However, the methods of obtaining the behavioral information are not limited to these examples. For example, the behavioral information may be obtained by first and second other exemplary methods described below.

In a first other exemplary method, behavioral information is obtained by using an artificial aneurysm that has been artificially created, an artificial heart connected to the artificial aneurysm, and an imaging device.

An artificial aneurysm is an artificial aneurysm that has occurred in an artificial blood vessel. The artificial blood vessel and the artificial aneurysm are created to mimic a human blood vessel and a human aneurysm that has occurred in the human blood vessel. The artificial aneurysm may be made of, for example, a rubber material, and a silicon rubber, a fluorocarbon rubber, or the like may be used, for example.

The artificial aneurysm may also be made of, for example, a silicon resin. As long as the artificial aneurysm is made of a flexible material, the material used for the artificial aneurysm is not limited to the above examples.

The artificial aneurysm is created utilizing image data obtained by an X-ray CT device or an MRI device as described above. This image data includes data on the human blood vessel and the aneurysm that has occurred in the blood vessel.

The artificial aneurysm is created based on digital imaging and communications in medicine (DICOM) data related to the image data obtained above.

An artificial heart is a device that performs the pumping function of a human heart. The artificial heart and the artificial aneurysm are connected, and the artificial heart's pumping function is activated to cause the artificial aneurysm to move in a pulsating manner. The behavioral information is obtained using this movement of the artificial aneurysm and the imaging device.

The imaging device is, for example, a camera device capable of capturing still images and videos. Furthermore, the imaging device may be a device capable of obtaining three-dimensional coordinates of a surface to be observed and information of a displacement in a three-dimensional space. Such an imaging device is capable of obtaining all the pieces of information which are three-dimensional coordinates on a surface of an observation target, a displacement in the three-dimensional space, a velocity in the three-dimensional space, and an acceleration in the three-dimensional space, by capturing a video for 1 second, 5 seconds, or 10 seconds.

Note that the time duration at which the imaging device performs imaging is not limited thereto, and may be other time duration. In this case, an X-ray CT device or an MRI device can also be used.

As described above, in the first other exemplary method, information on the three-dimensional coordinates on the surface of the artificial aneurysm and the displacement in the three-dimensional space are obtained by means of the imaging device capturing the video of the artificial aneurysm that pulsates. Behavioral information may be obtained based on any or all of the above pieces of information among the three-dimensional coordinates on the surface of the observation target and the displacement in the three-dimensional space.

In the first other exemplary method according to the first other example, such behavioral information can be obtained more easily than in the craniotomy described above, because the technique is less invasive.

In a second other exemplary method, a model animal having a blood vessel in which an aneurysm has occurred and the imaging device described above are used to obtain behavioral information. In this case, an X-ray CT device or an MRI device can also be used.

More specifically, the imaging device captures an image of a blood vessel with an aneurysm in the model animal to obtain the following pieces of information: three-dimensional coordinates in a three-dimensional space on the surface of the blood vessel with aneurysm in the model animal; and a displacement in the three-dimensional space. The behavioral information may be obtained based on any or all of the above pieces of information.

In the second other exemplary method, unlike a case involving a human as shown in the embodiment, a consent form and the like for the human subject of the case is not required. Additionally, since the surface of the blood vessel and aneurysm of the model animal can be patterned (for example, marked by spraying) for imaging, time evolution data of precise three-dimensional coordinates can be obtained.

Furthermore, data on the blood vessel and aneurysm of the model animal can be obtained at equal time intervals (for example, once every two weeks). This makes it easier to obtain behavioral information than in the embodiment.

The above method can be used to easily obtain a large number of pieces of behavioral information, and consequently, a large number of pieces of estimation information can be obtained. This is expected to improve the accuracy of the information about the wall.

Although the present embodiment describes the thickness of the blood vessel wall as the thickness of aneurysm wall 11 of cerebral aneurysm 10, the thickness of the blood vessel wall may be the thickness of the wall of the blood vessel that is an artery or a vein, as described above. For example, when the thickness of the wall is the thickness of the wall of the blood vessel that is an artery or a vein, the degree of stenosis of the artery or the vein is estimated using the blood vessel wall thickness estimation method and the like according to the embodiment.

In the foregoing embodiment, the constituent elements are constituted by dedicated hardware. However, the constituent elements may be realized by executing software programs corresponding to those constituent elements. Each constituent element may be realized by a program executing unit such as a CPU or a processor reading out and executing a software program recorded into a recording medium such as a hard disk or semiconductor memory.

Note that embodiments resulting from variations of the above embodiments arrived at by those skilled in the art, as well as embodiments resulting from optional combinations of elements and functions in the above embodiments are included within the present invention as long as the embodiments do not depart from the scope of the present invention.

### INDUSTRIAL APPLICABILITY

The wall thickness estimation method according to the present invention can be used in various applications, such as medical devices and medical methods.

### REFERENCE SIGNS LIST

10, 10a Cerebral aneurysm
11, 11a Aneurysm wall
12a Hole
20 Parent blood vessel
30, 30a Blood vessel model
31 Overall blood vessel model
40 Brain model
41 Left brain model
42 Right brain model
50 Skull model
100, 100a Wall thickness estimation device
110 First obtainer
110a Second obtainer
120 Generator
130 Outputter
140 First trainer
140a Second trainer
200 Display
300 Video information processing device
400 Video capturing device
500 Training device
610 Third obtainer
620 Constructor
1000, 1000a Wall thickness estimation system
2000 Model construction system
A Region
B Other subject
C Other subject
D Other subject
P Subject
p0, p1, p2, p3, p4, p5, p6, p7, p8, p9, p10, p11 Point
S102 First training step
S201 First obtaining step
S202 Generation step
S203 Outputting step
S301 Second obtaining step
S302 Second training step
S401 Third obtaining step
S402 First construction step
S403 Second construction step
S404 Third construction step

1. A wall thickness estimation method comprising:
obtaining behavioral information that is based on a video in which an organ wall or a blood vessel wall is captured using four-dimensional angiography, the behavioral information being numerical information about changes over time in a position of each of a plurality of predetermined points in the organ wall or the blood vessel wall;
generating estimation information using a model trained to take as an input an image indicating a physical parameter based on the behavioral information obtained in the obtaining and output an index indicating a thickness at each of the plurality of predetermined points in the organ wall or the blood vessel wall, the estimation information being information visualizing the thickness; and
outputting the estimation information generated in the generating.

2. The wall thickness estimation method according to claim 1, further comprising:
training the model using one or more datasets as training data, each of the datasets being constituted by a combination of (i) the image indicating the physical parameter based on the behavioral information at each predetermined point among the plurality of predetermined points and (ii) the index indicating the thickness at the predetermined point.

3. The wall thickness estimation method according to claim 2,
wherein in the training, the model is trained using machine learning.

4. The wall thickness estimation method according to claim 1,
wherein the estimation information is image information visualizing the thickness.

5. The wall thickness estimation method according to claim 1,
wherein the blood vessel wall is a wall of an arterial aneurysm or a varicose vein.

6. The wall thickness estimation method according to claim 1,
wherein the blood vessel wall is a wall of a cerebral aneurysm.

7. The wall thickness estimation method according to claim 1,
wherein the blood vessel wall is a blood vessel wall of an artery or a vein.

8. A non-transitory computer-readable recording medium having recorded thereon a computer program for causing a computer to execute the wall thickness estimation method according claim 1.

9. A training method comprising:
obtaining behavioral information that is based on a video in which an organ wall or a blood vessel wall is captured, the behavioral information being numerical information about changes over time in a position of each of a plurality of predetermined points in the organ wall or the blood vessel wall; and
training a model using, as training data, one or more datasets constituted by a combination of (i) an image indicating a physical parameter based on the behavioral information at each predetermined point among the plurality of predetermined points, the behavioral information being the behavioral information obtained in the obtaining, and (ii) an index indicating a thickness at the predetermined point among the plurality of predetermined points.

10. The training method according to claim 9,
wherein the video is obtained using four-dimensional angiography or a two-dimensional video capturing device.

11. A model construction method comprising:
obtaining the estimation information generated in the generating according to claim 1; and
constructing a blood vessel model including the blood vessel wall according to claim 1, the blood vessel model being constructed based on the thickness visualized by the estimation information obtained in the obtaining of the estimation information to cause the blood vessel wall included in the blood vessel model to exhibit a different form according to the thickness.

12. The model construction method according to claim 11,
wherein in the constructing of the blood vessel model, the blood vessel model is constructed such that the blood vessel wall exhibits a different color according to the thickness.

13. The model construction method according to claim 12,
wherein the blood vessel wall included in the blood vessel model constructed in the constructing of the blood vessel model is a wall of a cerebral aneurysm, and
the model construction method further comprises:
constructing a brain model into which the blood vessel model constructed in the constructing of the blood vessel model is incorporated.

14. The model construction method according to claim 13, further comprising:
constructing a skull model for containing the brain model constructed in the constructing of the brain model.

15. A wall thickness estimation device comprising:
an obtainer that obtains behavioral information that is based on a video in which an organ wall or a blood vessel wall is captured using four-dimensional angiography, the behavioral information being numerical information about changes over time in a position of each of a plurality of predetermined points in the organ wall or the blood vessel wall;
a generator that generates estimation information using a model trained to take as an input an image indicating a physical parameter based on the behavioral information obtained by the obtainer and output an index indicating a thickness at each of the plurality of predetermined points in the organ wall or the blood vessel wall, the estimation information being information visualizing the thickness; and
an outputter that outputs the estimation information generated by the generator.

16. A wall thickness estimation system comprising:
the wall thickness estimation device according to claim 15;
a video information processing device that obtains the video, generates the behavioral information, and outputs the behavioral information to the obtainer, and
a display that displays the estimation information output by the outputter.
